(19) United States
(12) Patent Application Publication
(10) Pub. No.: US 2025/0290919 A1
(43) Pub. Date: Sep. 18, 2025

(54) ULTRASENSITIVE SINGLE EXTRACELLULAR VESICLE DETECTION USING HIGH THROUGHPUT DROPLET DIGITAL ENZYME-LINKED IMMUNOSORBENT ASSAY

(71) Applicant: THE TRUSTEES OF THE UNIVERSITY OF PENNSYLVANIA, Philadelphia, PA (US)

(72) Inventors: David Aaron ISSADORE, Philadelphia, PA (US); Zijian YANG, Palo Alto, CA (US)

(21) Appl. No.: 18/861,287

(22) PCT Filed: May 4, 2023
(86) PCT No.: PCT/US2023/066593

§ 371 Date: Oct. 29, 2024

Related U.S. Application Data
(60) Provisional application No. 63/364,281, filed on May 6, 2022.

(51) Int. Cl. G01N 33/533; B01L 3/00; G01N 33/535

(52) U.S. Cl. CPC G01N 33/533; B01L 3/502715; B01L 3/502784; G01N 33/535; G01N 2800/00

(57) ABSTRACT

Extracellular vesicles (EVs) have attracted enormous attention for their diagnostic and therapeutic potential. However, it has proven challenging to achieve the sensitivity to detect individual nanoscale EVs, the specificity to distinguish EV subpopulations, and a sufficient throughput to study EVs amongst an enormous background. To address this fundamental challenge, we developed a droplet-based optofluidic platform to quantify specific individual EV subpopulations at high throughput. The key innovation of our platform is parallelization of droplet generation, processing, and analysis to achieve a throughput (˜20 million droplets/minute) more than 100× greater than typical microfluidics. We demonstrate that the improvement in throughput enables EVs detection at a limit of detection=9EVs/μL, a >100× improvement over gold standard methods. Additionally, we demonstrate the clinical potential of this system by detecting human EVs in complex media. Building on this work, we expect this technology will allow accurate quantification of rare EV subpopulations for broad biomedical applications.

### CROSS-REFERENCE TO RELATED APPLICATIONS

The present application claims priority to and the benefit of U.S. patent application No. 63/364,281, “Ultrasensitive Single Extracellular Vesicle Detection Using High Throughput Droplet Digital Enzyme-Linked Immunosorbent Assay” (filed May 6, 2022). All foregoing applications are incorporated herein by reference in their entireties for any and all purposes.

### GOVERNMENT RIGHTS

This invention was made with government support under W81XWH-19-2-0002 awarded by the Defense Advanced Research Projects Agency, and CA236653 awarded by the National Institutes of Health. The government has certain rights in the invention.

### TECHNICAL FIELD

The present disclosure relates to the field of microfluidics and to the field of detection of extracellular vesicles.

### BACKGROUND

Extracellular vesicles (EVs) have attracted enormous attention for their diagnostic and therapeutic potential. However, it has proven challenging to achieve the sensitivity to detect individual nanoscale EVs, the specificity to distinguish EV subpopulations, and a sufficient throughput to study EVs amongst an enormous background. Accordingly, there is a long-felt need in the art for improves systems and methods for EV detection.

### SUMMARY

In meeting the described long-felt needs, the present disclosure provides a method, comprising: contacting (i) a sample comprising a plurality of extracellular vesicles (EVs) and (ii) a detectable capture modality complementary to a target EV so as to form a first product; contacting the first product with a detection modality that associates with the target EV so as to form a second product that comprises a plurality of the detectable capture modalities; and within a plurality of droplets, contacting the second product and a substrate that is reactive with the detection modality to produce a detectable signal indicative of the presence of the second product within the droplet; and optically interrogating the plurality of droplets.

Also provided is a system, the system being configured to perform the method of the present disclosure, e.g., the method of any one of Aspects 1-24.

Further disclosed is a system, comprising: a droplet generation section configured to form a plurality of droplets having therein a substrate and a final product comprising an EV and reactive with the substrate to produce a detectable signal indicative of the presence of the final product within the droplet; an incubation section in fluid communication with the droplet generation section and configured to communicate therein the droplets, the incubation section operable to provide a residence time for the droplets sufficient to give rise to the detectable signal indicative of the presence of the final product within the droplet; and a detection section, the detection section configured to communicate therein the plurality of droplets through at least one channel of a microfluidic device, illuminate the droplets with a first time-domain modulated sequence of flashes from a first light source, and capture a plurality of images of the droplets.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

In the drawings, which are not necessarily drawn to scale, like numerals may describe similar components in different views. Like numerals having different letter suffixes may represent different instances of similar components. The drawings illustrate generally, by way of example, but not by way of limitation, various aspects discussed in the present document. In the drawings:

FIG. 1A-1C. Illustration of high throughput droplet based extracellular vesicle assay (DEVA).

FIG. 2A-2D. Illustration of theoretical calculation demonstrating the benefits of more beads in DEVA.

FIG. 3A-3E. Illustration of high throughput detection schematic for DEVA.

FIG. 4A-4C. Characterization of human neuron EVs for DEVA.

FIG. 5A-5H Benchmarking and characterization of DEVA.

FIG. 6. A photograph of the experimental setup of our DEVA platform

FIG. 7. Excitation laser characterization for DEVA

FIG. 8A-8E. DEVA fluorescence characterization.

FIG. 9A-9D. High throughput droplet generation enabled by parallelized flow focusing generators.

FIG. 10. SEM images of DEVA.

FIG. 11. Schematics on determining the ratio of CD81+EVs among the total human neuron EVs using Qubit protein assays.

FIG. 12A-12B. DEVA optimization on reagent concentration.

FIG. 13A-13B. Supplementary fluorescent microscopic images.

FIG. 14A-14B. Human neuron EVs characterization on conventional tools.

FIG. 15A-15B. NTA analysis on isolated EVs.

FIG. 16A-16D. Feasibility of measuring endogenous EVs in human plasma using DEVA.

### DETAILED DESCRIPTION OF ILLUSTRATIVE EMBODIMENTS

The present disclosure may be understood more readily by reference to the following detailed description of desired embodiments and the examples included therein.

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing. All publications, patent applications, patents and other references mentioned herein are incorporated by reference in their entirety. The materials, methods, and examples disclosed herein are illustrative only and not intended to be limiting.

The singular forms “a,” “an,” and “the” include plural referents unless the context clearly dictates otherwise.

As used in the specification and in the claims, the term “comprising” may include the embodiments “consisting of” and “consisting essentially of.” The terms “comprise(s),” “include(s),” “having,” “has,” “can,” “contain(s),” and variants thereof, as used herein, are intended to be open-ended transitional phrases, terms, or words that require the presence of the named ingredients/steps and permit the presence of other ingredients/steps. However, such description should be construed as also describing compositions or processes as “consisting of” and “consisting essentially of” the enumerated ingredients/steps, which allows the presence of only the named ingredients/steps, along with any impurities that might result therefrom, and excludes other ingredients/steps.

As used herein, the terms “about” and “at or about” mean that the amount or value in question can be the value designated some other value approximately or about the same. It is generally understood, as used herein, that it is the nominal value indicated ±10% variation unless otherwise indicated or inferred. The term is intended to convey that similar values promote equivalent results or effects recited in the claims. That is, it is understood that amounts, sizes, formulations, parameters, and other quantities and characteristics are not and need not be exact, but can be approximate and/or larger or smaller, as desired, reflecting tolerances, conversion factors, rounding off, measurement error and the like, and other factors known to those of skill in the art. In general, an amount, size, formulation, parameter or other quantity or characteristic is “about” or “approximate” whether or not expressly stated to be such. It is understood that where “about” is used before a quantitative value, the parameter also includes the specific quantitative value itself, unless specifically stated otherwise.

Unless indicated to the contrary, the numerical values should be understood to include numerical values which are the same when reduced to the same number of significant figures and numerical values which differ from the stated value by less than the experimental error of conventional measurement technique of the type described in the present application to determine the value.

All ranges disclosed herein are inclusive of the recited endpoint and independently of the endpoints (e.g., “between 2 grams and 10 grams, and all the intermediate values includes 2 grams, 10 grams, and all intermediate values”). The endpoints of the ranges and any values disclosed herein are not limited to the precise range or value; they are sufficiently imprecise to include values approximating these ranges and/or values. All ranges are combinable.

As used herein, approximating language may be applied to modify any quantitative representation that may vary without resulting in a change in the basic function to which it is related. Accordingly, a value modified by a term or terms, such as “about” and “substantially,” may not be limited to the precise value specified, in some cases. In at least some instances, the approximating language may correspond to the precision of an instrument for measuring the value. The modifier “about” should also be considered as disclosing the range defined by the absolute values of the two endpoints. For example, the expression “from about 2 to about 4” also discloses the range “from 2 to 4.” The term “about” may refer to plus or minus 10% of the indicated number. For example, “about 10%” may indicate a range of 9% to 11%, and “about 1” may mean from 0.9-1.1. Other meanings of “about” may be apparent from the context, such as rounding off, so, for example “about 1” may also mean from 0.5 to 1.4. Further, the term “comprising” should be understood as having its open-ended meaning of “including,” but the term also includes the closed meaning of the term “consisting.” For example, a composition that comprises components A and B may be a composition that includes A, B, and other components, but may also be a composition made of A and B only. Any documents cited herein are incorporated by reference in their entireties for any and all purposes.

Extracellular vesicles (EVs) are a diverse set of lipid bound nanomaterials (1), which can be found circulating in blood and carry various molecular cargo that can be representative of their cells of origin (2, 3). EVs have been discovered to play an important role in intercellular communication (4) and to have enormous potential as biomarkers for a wide range of biomedical applications (5-11). Because EVs are heterogenous, there has been an effort to measure EVs with single particle resolution. This technological push is analogous to the development of single cell analysis that fueled biological discovery in the last decade (12-16). Many of the proposed techniques are based on imaging (17-20). While imaging has been successful in resolving individual EVs, it is fundamentally limited to a number of particles (<10⁴) several orders of magnitude less than the quantity of EVs found in clinical samples (10¹⁰ EVs/mL, 10⁸ EVs in 10 μL of plasma) (21). Digital droplet techniques, wherein single EVs are loaded into droplets and either barcoded for downstream next generation sequencing (NGS) analysis or digital enzyme-linked immunosorbent assay (dELISA) based fluorescence detection, have been particularly successfully addressing this challenge (22-25). However, a single platform has been unable to achieve the sensitivity to detect individual EVs, the specificity to distinguish particular EV subsets based surface protein expression, at throughputs relevant to study rare EV subpopulations.

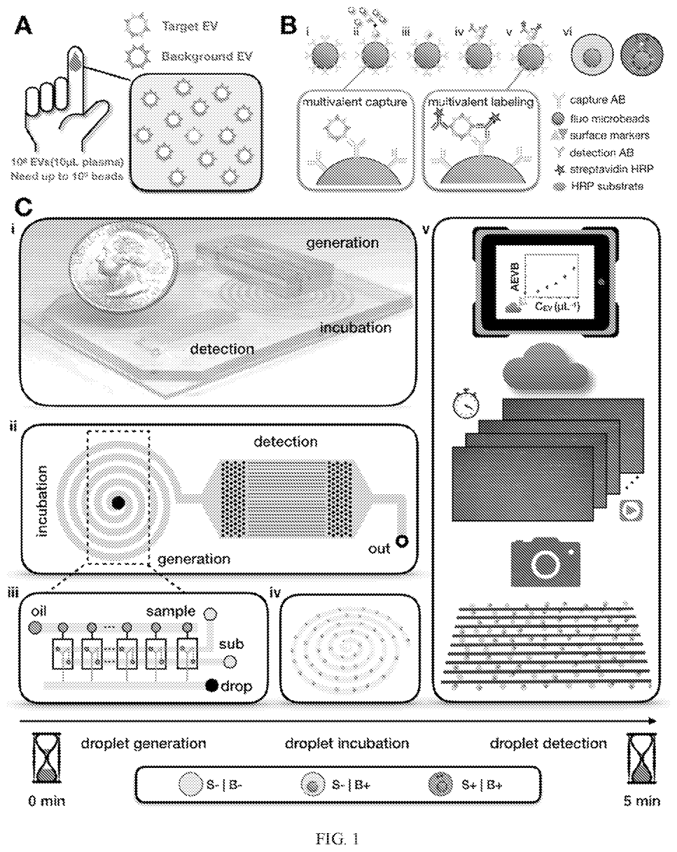

In this work, we developed a high throughput digital assay to quantify rare EV sub-populations based on their expression of surface proteins amongst a large background of EVs (26-28) (FIG. 1A). We call our approach DEVA (Droplet based Extracellular Vesicle Analysis). DEVA's ultra-sensitivity and robustness to complex background comes primarily from two interrelated aspects of the technology: a) The multiple proteins expressed on a targeted EV surface allows for specific capture and labeling on microbeads. Due to this specificity, <0.03% of our beads resulted in a false positive signal, 8-40× lower than typical dELISAs (26-28) (Table 1). b) At such a low false positive rate, we found that our limit of detection (LOD) was Poisson noise constrained until the bead number was increased to N_{bead}=O (10⁶), which corresponded to N_{drop}=O (10⁷) to ensure a digital distribution of beads within droplets (Table 2). Accordingly, we developed a platform to measure beads at a high throughput (˜20 million droplets/minute) by parallelizing droplet generation, processing, and analysis, achieving a throughput >100× greater than typical in microfluidic systems using only accessible optics (<$1,000) and soft-lithography fabrication. We evaluated our technology by first quantifying human neuron derived EVs spiked into PBS and achieved a LOD=9 EVs/μL, a>100× improvement over standard single EV characterization methods (29). Moreover, we directly demonstrated the value of the throughput of our system by showing that the LOD of DEVA improved with an increase in the number of beads, up to 10⁶ beads. Additionally, we evaluated the application of this system for use in clinical samples by quantifying human neuron EVs spiked in complex media, with a background EVs matched to that of 2 μL of human blood and found an LOD=11 EVs/μL.

### Single EV Detection on DEVA

Our DEVA assay uses fluorescent paramagnetic microbeads (d=5.4 μm) functionalized with a capture antibody to target EVs subpopulations based on their expression of a particular surface protein (FIG. 1B). We label the beads with a fluorescence signal for two key reasons. First, it allows us to calculate the ratio Average EV per Bead (AEVB) accurately, without having to make an assumption about the number of beads that have flowed through our system. Secondly, it provides a convenient means to multiplex our system, wherein the color of the microbead can indicate the capture antibody (30). The microbeads are first incubated with the sample to capture target EVs. After magnetic separation and washing, captured EVs are labelled with a biotinylated labelling antibody, followed by a streptavidin-HRP enzyme, to form an enzyme-linked immunocomplex. After washing to remove unbound antibodies and enzyme, the beads are mixed with a fluorescence enzyme substrate (ThermoFisher) and loaded into d=20 μm aqueous droplets suspended in oil (Bio-Rad) using parallelized droplet generators (FIG. 1C). The droplets are incubated for 5 min on-chip to allow the immunocomplexes to generate a fluorescent signal. A fluorescent droplet corresponds to a single EV that is positive for both the protein targeted by the capture antibody and the labelling antibody. Each droplet is inspected using our microdroplet megascale detector (μMD) platform in two fluorescence channels (30), one channel to determine whether the droplet contains a fluorescent bead and the other to interrogate if the droplet is fluorescent. The ratio of beads that have captured an EV versus the total number of beads is the AEVB. To ensure the assay is in the digital regime, i.e. there is one or zero EVs per bead and one or zero bead per droplet, we use >10× as many beads as EVs and >10× as many droplets as beads.

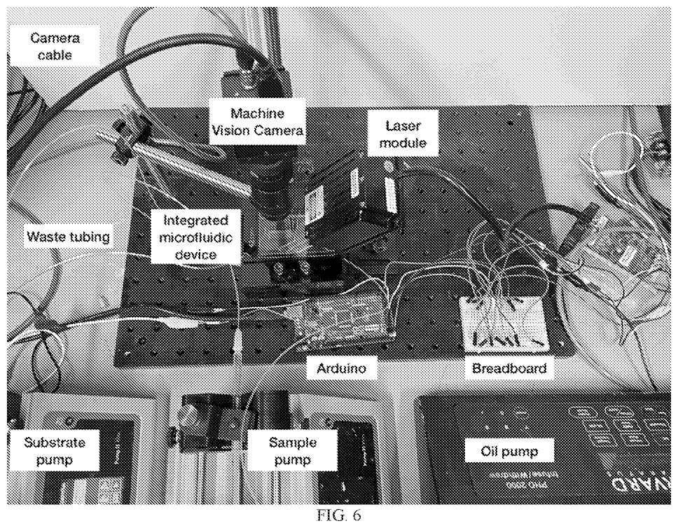

We perform high throughput fluorescence detection using a digital camera by modulating the light source in time with a pseudorandom sequence at a rate >10× the frame rate of the camera, encoding the droplet streak with a pattern that allows it to be resolved using correlation based detection amongst neighboring droplets (30). Downstream of the delay line, droplets flow through a detection region that consists of 90 parallelized microfluidic channels, where their fluorescence is measured using two time-domain modulated laser diodes (31) and a machine vision camera (Grasshopper) (FIG. 1C, FIG. 6). In this implementation, a blue laser and a green laser module (λ_{exBlue}=457 nm, λ_{exGreen}=528 nm, Techhood) were each modulated by an independent pseudorandom sequence, provided by a microcontroller (Arduino), to excite the fluorophores on microbeads and HRP substrate respectively. Each pseudorandom sequence was an algorithmically generated 63-bit maximum length sequence (MLS), chosen to be minimally autocorrelated with itself and cross correlated with one another (31). Videos from the camera are processed by either a local computer, or on the cloud. Additional information can be found in U.S. Pat. No. 10,809,176, the entirety of which is incorporated herein by reference for any and all purposes.

### The Benefit of High Throughput Digital EV Detection

We found that increasing the number of beads until N_{bead}=O (10⁶) was a key factor in achieving the LOD and the dynamic range (DR) of our DEVA platform. The LOD of a digital ELISA is a function of both the background level AEWB_{b}, the AEVB for no target EVs and the standard deviation σ_{AEB}_{_{b}} across independent measurements of the background level, such that LOD=AEB_{b}+3σ_{AEB}_{_{b}}. We were able to achieve a background level of AEB_{b}=2.6×10⁻⁴, compared with that in typical digital ELISA(AEB_{b}=[2×10⁻³, 2×10⁻²]) (Table 1) (27, 32). With such a low false positive rate, if we had used 25,000 beads, as is typical in digital assays, we would only measure N_{b}=7 false positive events. With only 7 false positive events, the σ_{AEB}_{_{b}} of the background level from shot noise alone is

$${σ}_{{AEB}_{b}} = \frac{\sqrt{{N}_{b}}}{{N}_{b}} = 38 \% ,$$

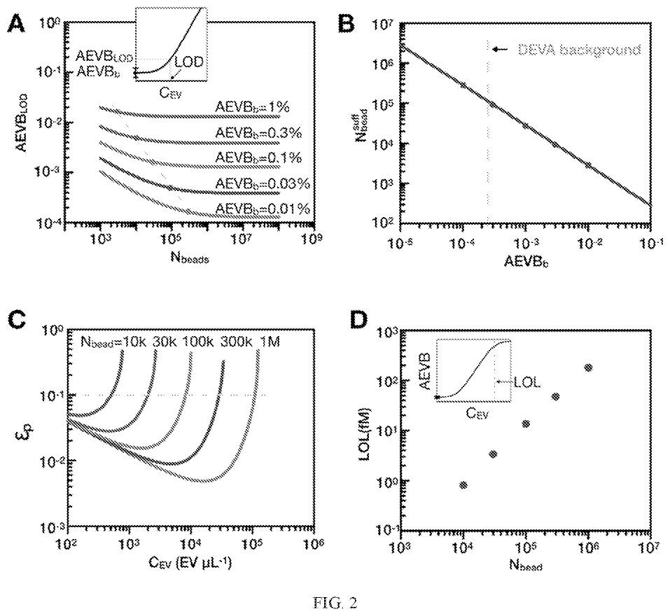

which would dominate the LOD if we assume the operational variance between experiments was σₑₓₚ=5%. Given the dependence of the variance of AEVB on the number of beads N_{bead}, the LOD can be improved by increasing the number of beads in an experiment until Poisson noise is no longer greater than the operational variance (FIG. 2A). As conventional bead-based digital ELISA detects beads within wells on a statically micromachined chip, the total number of beads in an experiment is usually between several thousands to tens of thousands (22, 26, 32, 33), which fundamentally sets the background level AVEB_{b} to be ˜0.2%, below which it would make no further improvement to the LOD. For our platform, we found that LOD could be improved by increasing beads up to N_{bead}=O (10⁶), resulting in N_{drop}=20N_{bead}, 2×10⁷ (FIG. 2B). To process N_{drop}=2×10⁷ at typical throughputs of flow cytometers or microfluidic systems ϕ_{typ}=10³ droplet/sec, it would take approximately 5 hours to process a single experiment.

In addition to improving LOD, increasing the bead number also improve the dynamic range (DR) for DEVA. The upper value of the DR of a digital assay is limited by partitioning error εₚ, which becomes significant when the number EVs becomes similar to the number of beads N_{beads}˜N_{EVs}, making it increasingly probable that there will be more than one EV bound to each positive bead (λ>1, λ is the average number of EVs per bead). This partitioning error (εₚ) is modeled based on the binomial process (34) and is expressed as

$${ε}_{p} = \frac{1}{λ} ln ( \frac{{e}^{- λ} + {z}_{c} {σ}_{E}}{{e}^{- λ} + {z}_{c} {σ}_{E}} ) ,$$

where Z_{c}=1.96 for 95% confidence interval and σ_{E}=\sqrt{e^{-λ}(1−e^{-λ})/N_{bead}} (FIG. 2C). Incorporating more beads into our assay thus improve the LOL, by keeping partitioning error low (εₚ<10% of final readout) for increasing quantities of target EV (FIG. 2D). Leveraging our system's high throughput to measure large numbers of beads, both the LOD and the LOL can be improved, resulting in an expanded dynamic range for target EVs. From a user's perspective, if the number of EVs falls below the LOD or over the LOL, the system would report accordingly.

### Microfluidic Integration of Our High Throughput Bead Analysis

Building on our previous work on high throughput detection of droplets using time-domain-encoded optofluidics (30), we developed a high throughput single EV analysis platform by parallelizing droplet generation, processing, and analysis to achieve a throughput >100× greater than typical in microfluidic systems (FIG. 1C). We updated our previous platform to be applied to DEVA in two main aspects. First, we used a digital camera, which uses a global shutter instead of rolling shutter (35), to prevent uncertainty in the phase of the time domain modulated fluorescent streaks across the image's 90 parallelized microfluidic channels. Second, we optimized the image processing algorithms and applied GPU acceleration for image analysis. The resulting calculations for each experiment decreased from 1000 min to 5 min.

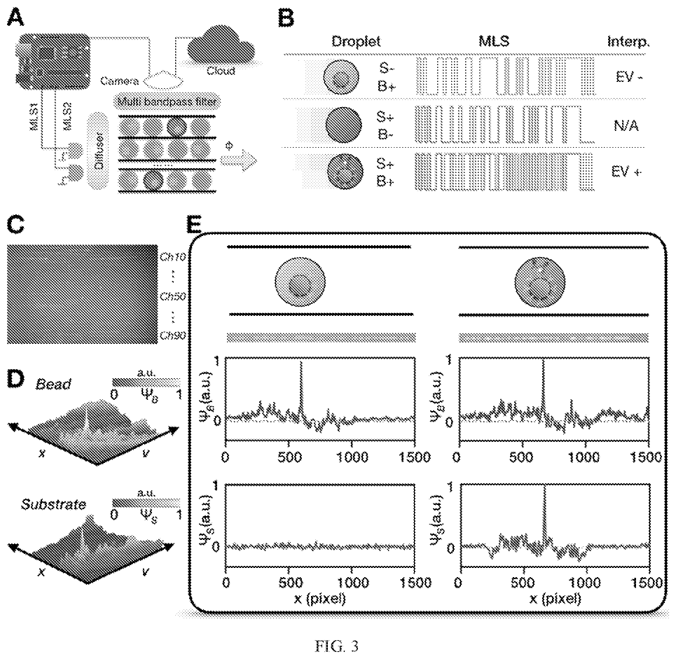

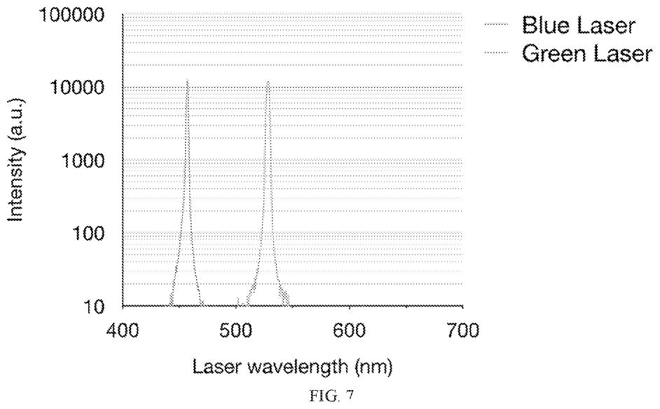

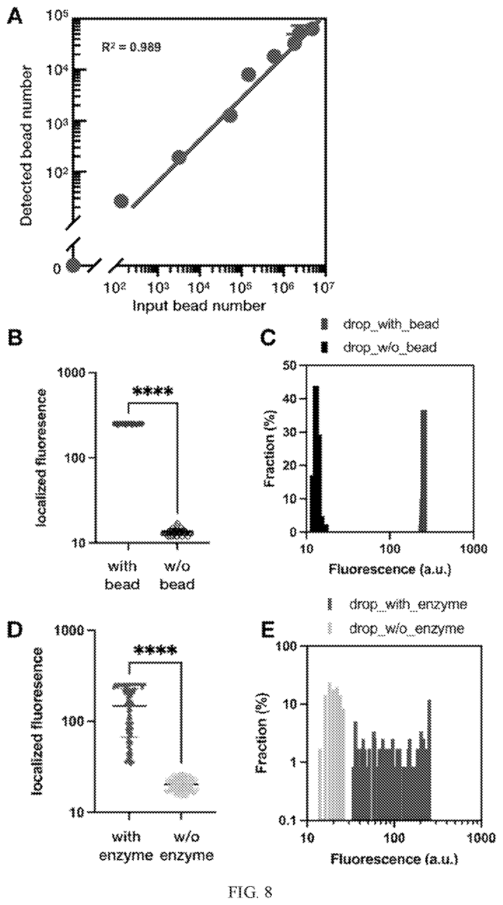

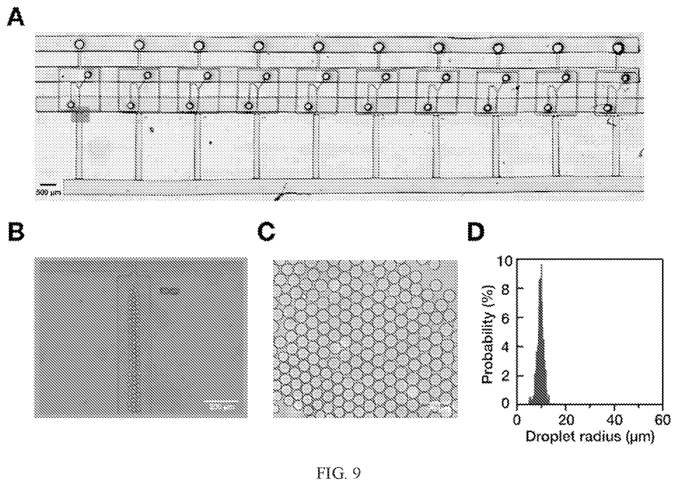

The workflow of our detection modality is described below. A machine vision camera (Grasshopper3) with a macro lens (Computar) records the fluorescence signal from all 90 microfluidic channels for image analysis (FIG. 3A, FIG. 3C). The dimensions of each detection channel is 40×45 μm², and the entire detection region occupies a space of 15×9 mm². A multiband pass filter (λ_{cwl}=485±10 nm, 559.5±12.5 nm; Semrock) is incorporated between the camera and the device to block scattered excitation light. The fluorescence signal from bead and substrate are chosen to have spectrally separated absorption spectra so they can be independently excited by our blue laser (λ_{exBlue}=457 nm) and the green laser (λ_{exGreen}=528 nm) (FIG. 7), respectively. Each laser is modulated by a unique 63-bit MLS sequence (FIG. 3B) so they can be resolved via correlation detection (FIG. 3D) (31, 36). The velocity of the droplets and exposure time of our camera (45 ms) is chosen to match the 63-bit MLS pattern with a droplet's imaged streak of 5 mm, which can be resolved by our optical system that has a resolution of 30 μm. A threshold is chosen to identify beads and positive droplets from the correlation signal of both the bead and fluorescence channel, by choosing a threshold that equals 3 standard deviations above the noise floor in the correlation signal. Additionally, we analyze the fluorescence signal of droplets with beads versus those without, and droplets with an enzyme versus those without, in FIG. 8. We find that we can separate droplets with beads versus those without with an AUC=1 (N=41 droplets) and droplets with and without enzyme with an AUC=1 (N=118 droplets). Further details of our multidimensional correlation analysis were reported previously (30). An example of raw imaging data, and the corresponding correlation analysis, of a typical signal of a droplet containing a microbead that has and has not captured a single EV appear in FIG. 3E. To characterize this approach, we spiked known quantities of fluorescent microbeads into our detection platform and quantified the number of measured beads (FIG. 8) (R²=0.989). To match the throughput of our fluorescence detection, we integrated high throughput droplet generation (20 million droplets/min, d=19.5 μm) by parallelizing 10 flow focusing droplet generators onto a single chip (FIG. 9). We used a three-dimensional ladder geometry to uniformly distribute fluids to each droplet generator and collect each generator's output (35).

### Development and Characterization of Assay Conditions for Ultrasensitive Single EV Detection

As a model system to evaluate DEVA, we used EVs isolated from a human neuronal cell line (37) (SI). From this cell culture media, we isolated EVs using a commercial total-exosome isolation kit (ThermoFisher). We chose human neuron-derived EVs because neuronal EVs participate in neuron-glial communication, neuroinflammation, and propagation of pathogenic proteins such as amyloid-beta (38-40). These processes help create the neurodegenerative microenvironment of evolving traumatic brain injury pathology, along with other chronic neurologic disease pathologies (9). Moreover, as EVs can cross the blood-brain-barrier while remaining intact, they have great potential as biomarkers to monitor central nervous system injury and recovery (41).

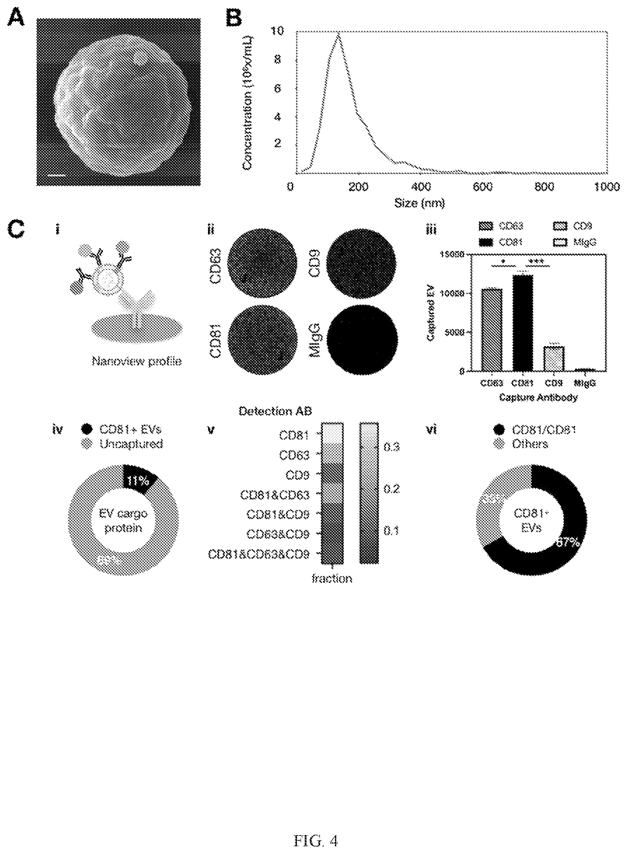

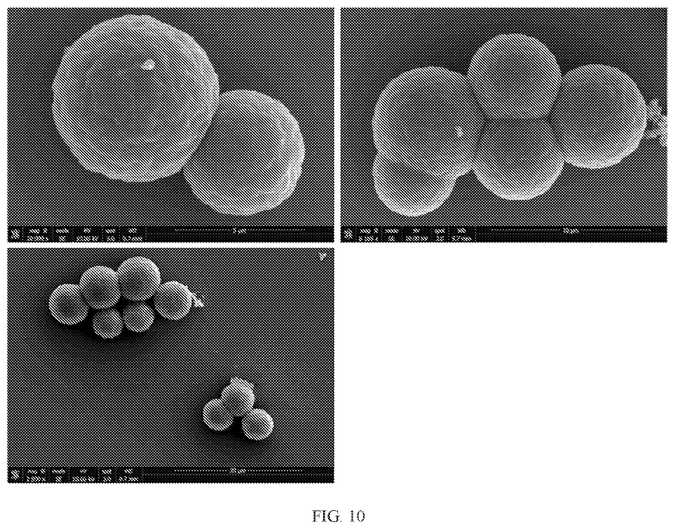

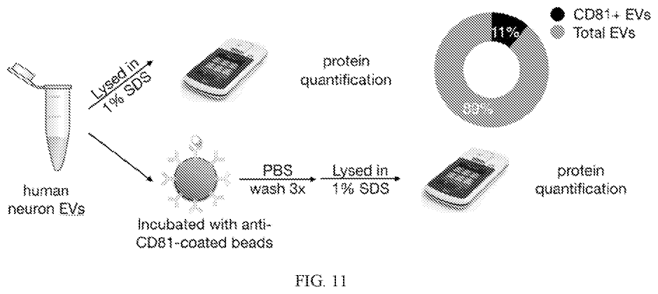

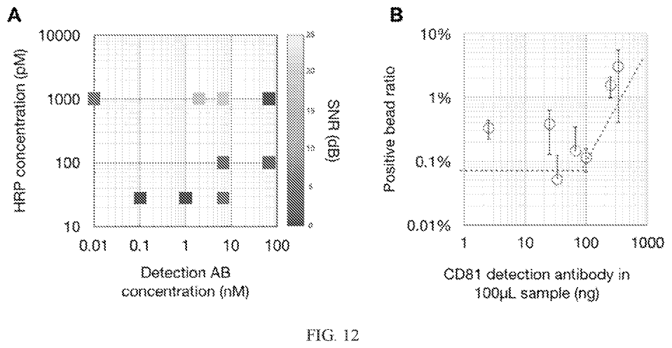

We characterized the EVs from this model system using Scanning Electron Microscopy (SEM), nanoparticle tracking analysis (NTA), and Nanoview which is a commercial platform for EV concentration, phenotype, and biomarker colocalization analysis. SEM demonstrated that, qualitatively, our antibody functionalized magnetic microbeads were capturing single EVs (FIG. 4A, FIG. 10). NTA analysis was used to quantify the concentration of our cell culture derived EVs (1.4×10⁹ EV/mL) and their size distribution (d=155 nm) (FIG. 4B). We used Nanoview to determine which antibody to use to capture and to label EVs in our model system. With Nanoview, we quantified the heterogeneous protein expression on the EVs' surface. In this assay, EVs are first captured on antibody coated chips, then labelled with antibodies (CD9/CD63/CD81) conjugated with different fluorophores (FIG. 4C). By counting the fluorescent events in each channel, we found that CD81 showed significant higher expression than CD63 or CD9 on our human neuron derived EVs (p<0.05, p<0.0001), which was also significantly higher than the MIgG negative control (p<0.0001). Hence, we chose to functionalize our beads with anti-CD81 as capture antibody in DEVA. After EV capture, we used 1% SDS to lyse the captured CD81⁺ EVs and compared their protein cargo to the total EV input. The result showed 11% (9.6%-12.4%) of all EVs expressed at least one CD81 marker on their surface (FIG. 11). Among CD81⁺ EVs, we found that 67% (64.3%-69.8%) of EVs co-expressed another copy of CD81 that could be labelled with a fluorophore-conjugated antibody. Consistent with the observation of others, we found heterogeneity among EV's surface protein “pan-EV” markers even though they were derived from the same cell line (18, 42, 43). We chose to use CD81 for capture and labelling, rather than neuron specific surface markers, because this protein could be characterized using established Nanoview assay kits, allowing us to quantify and benchmark the performance of DEVA. To improve the performance of DEVA assay required optimization in a multi-dimensional parameter space, including the concentration of several reagents and blocking conditions. For instance, we evaluated the concentration of the labelling antibody and HRP enzyme to balance tradeoff between nonspecific and specific labeling (FIG. 12). The concentration of labelling antibody (6.6 nM) and HRP (1 nM) achieved the current performance that allowed us to validate our technology, and we believe that even better performance may be possible by further optimization.

### Evaluation of the Performance of DEVA for Single EV Analysis

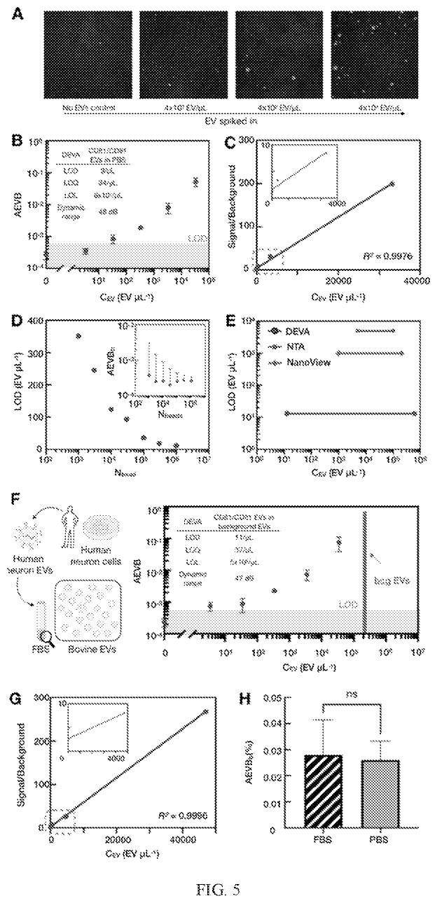

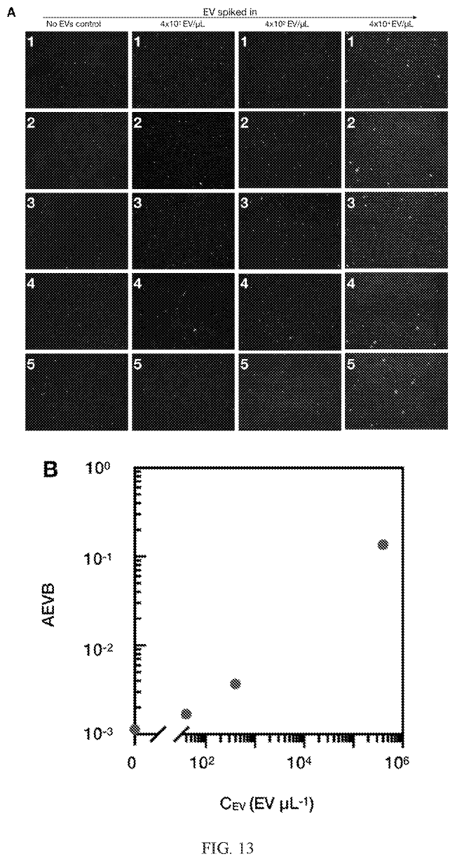

We initially evaluated the performance of DEVA by quantifying human neuron EVs spiked into PBS at known concentrations. We first evaluated the assay qualitatively by examining the droplets under a microscope (Leica) (FIG. 5A, FIG. 13). Assays run with increasing quantities of EVs showed an increase in the number of droplets that contain beads and that fluoresced red, each of these instances indicating a single detected targeted EV. Subsequently, we quantitatively analyzed the performance of DEVA by measuring the response of our μMD system to a serial dilution of human neuron EVs from 0−10⁵ EV/μL spiked into 100 μL PBS (FIG. 5B). The background level, when no EVs were spiked in, was AEB_{b}=0.026%, which is 8-40 times lower compared with typical dELISA (28, 33). We quantified the LOD of DEVA for detecting targeted EVs in PBS as LOD=9 EVs/μL. Within the dynamic range (DR, 9−5×10⁵ EVs/μL), DEVA showed good linearity (FIG. 5C, R²=0.9976).

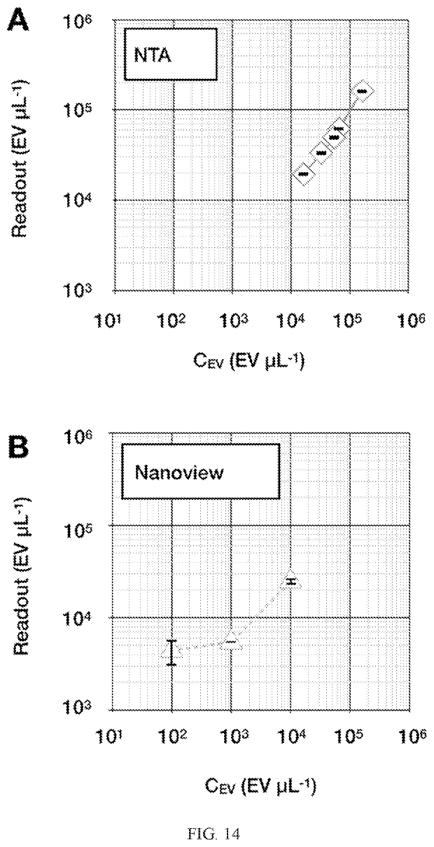

We directly evaluated the role of the number of beads used in a DEVA assay to reduce the Poisson noise in the measurement and thus improve the LOD. Using the same assay conditions, we calculated the AEVB_{b} by analyzing 10³ to 10⁶ beads. As expected, analyzing more beads decreased the standard deviation of the measurement of the response to a blank input AEVB_{b}, which decreased the LOD of DEVA (FIG. 5D). We also compared the performance of DEVA with conventional EV characterization platforms NTA and Nanoview, using aliquots of the same samples processed by our μMD (FIG. 14). NTA is a common tool for EV quantification and has been shown to analyze surface proteins (29). We found, consistent with the literature, the LOD for NTA is ˜ [symbol] (10⁴ EV/μL) with limited DR (10⁴−2×10⁵ EV/μL). Nanoview provides a multiplexed surface protein profiling of individual EVs. We quantified the LOD of Nanoview to be ˜ [symbol] (10³ EV/μL) and the DR to be 2.3 log range, similar with their commercial characterization (2.8 log). In comparison to these technologies, DEVA showed a 100× better LOD and a 200× better DR for single EV characterization (FIG. 5E).

### Characterization of Background Invariance of DEVA

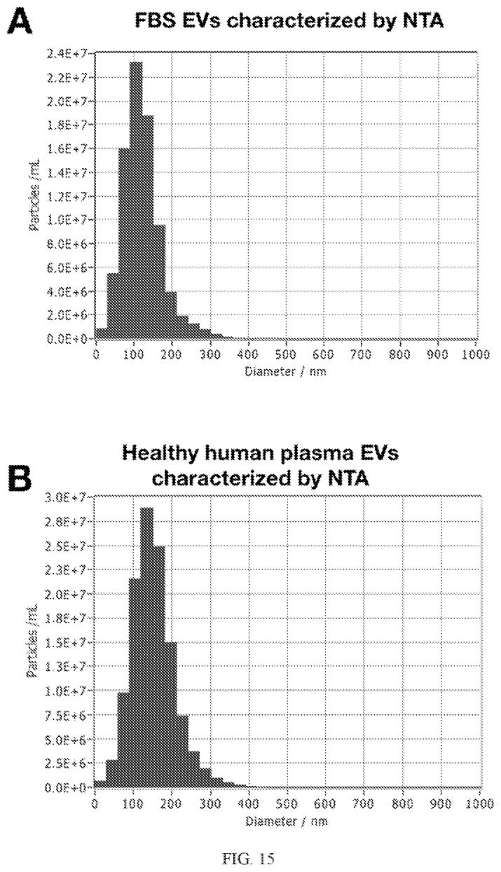

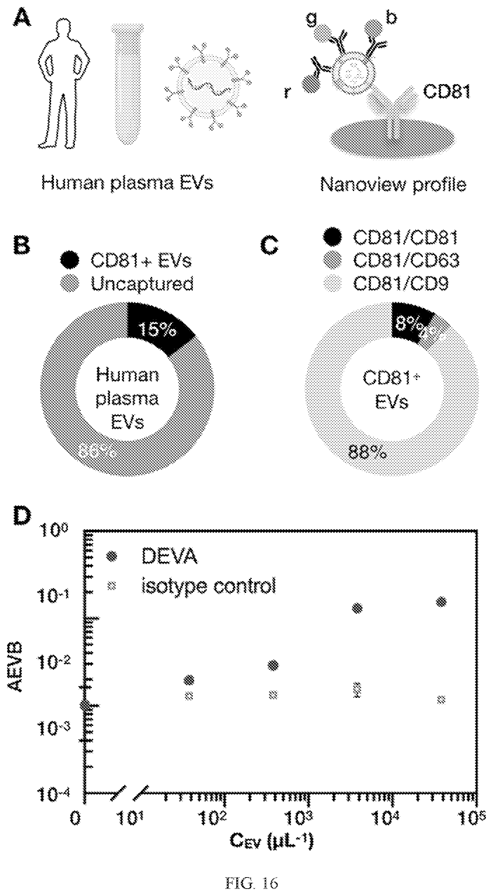

We evaluated the performance of DEVA to quantify rare EVs in the presence of complex media. To this end, we spiked human neuron EVs into fetal bovine serum (FBS) that contained 2×10⁷ bovine EVs as background (FIG. 15), similar to the number of human EVs present in a 2 μL blood from a typical finger prick. We chose FBS because it models a complex background but does not contain any EVs positive for hum-CD81 (24, 30). We analyzed the performance of DEVA by measuring the response of our μMD system to a serial dilution of human neuron EVs from 0-10⁵ EV/μL spiked into 100 μL FBS (FIG. 5F). The LOD of DEVA in complex media is 11 EVs/μL. DEVA demonstrated good linearity within the dynamic range (FIG. 5G, R²=0.9996). We found that the bovine EV background did not have a significant effect on the LOD of DEVA compared to measurements with no background in PBS (FIG. 5H), demonstrating the value of this technology to be applied to detecting rare EVs in clinical samples. Additionally, we demonstrated the feasibility of quantifying endogenous EVs in human plasma, using isotope antibodies for capture and labelling as a negative control (FIG. 16).

Our DEVA platform, with its high sensitivity (LOD=9 EVs/μL), and its high droplet throughput (20M droplets/min), make it possible to quantify sparse EV subpopulations in complex media. The key to our device's sensitivity is its processing of tens of millions of droplets, and its capability to scale its processing rate only at a cost in computation. While in this first demonstration we performed a single-plex assay to quantify EVs based on CD81/CD81 expression, we can combine our droplet throughput and a multicolor detection approach to analyze multiple EV subpopulations simultaneously. Multiplexing can be increased by running assays in parallel, using microbeads barcoded with distinct ratios of concentrations of multiple dyes to antibody that they are functionalized with, as has been done by Luminex for non-digital ELISA (44). Alternatively, or in combination, the sample can be divided to be mixed with different panels of microbeads in individual sets of channels of the n=90 detection channels. By combining these two approaches, it is possible for DEVA to achieve >100 multiplexed EV assays. Recently, a growing number of studies has shown the clinical value of measuring <8 of EV subpopulations (5, 7, 45-49). In prior work, a duplex proteins assay provides a starting point (30). In addition to EVs, our approach can be applied to other nanoscale objects, such as mitochondria to study their dysfunction in Alzheimer's disease (50) or HIV virus to study transfection mechanisms (51). Additionally, we are encouraged by recent advances in digital assays, which either obviate the need for droplets by replacing enzymatic amplification with rolling circle amplification directly on beads or obviate the need for microfluidics by using beads that template droplets (28, 52). These techniques, incorporated with DEVA, could provide a further 10× improvement in throughput and using a simpler implementation, which can be leveraged to increase multiplexing, further improve sensitivity, and allow point-of-care use. In addition to improving the LOD, the throughput of our system can be leveraged to increase the LOL by reducing partitioning error at high concentrations of EVs, resulting in an increased dynamic range. By applying high throughput digital droplet detection to single EV analysis, the μMD would allow the ultrasensitive, multiplexed EV detection in complex media, opening a broad range of new possibilities for clinical diagnostics and biological inquiry.

### REFERENCES

1. R. Kalluri, V. S. LeBleu, The biology, function, and biomedical applications of exosomes. Science (80-.). 367 (2020),, doi: 10.1126/science.aau6977.
2. B. Costa-Silva, N. M. Aiello, A. J. Ocean, S. Singh, H. Zhang, B. K. Thakur, A. Becker, A. Hoshino, M. T. Mark, H. Molina, J. Xiang, T. Zhang, T. M. Theilen, G. García-Santos, C. Williams, Y. Ararso, Y. Huang, G. Rodrigues, T. L. Shen, K. J. Labori, I. M. B. Lothe, E. H. Kure, J. Hernandez, A. Doussot, S. H. Ebbesen, P. M. Grandgenett, M. A. Hollingsworth, M. Jain, K. Mallya, S. K. Batra, W. R. Jarnagin, R. E. Schwartz, I. Matei, H. Peinado, B. Z. Stanger, J. Bromberg, D. Lyden, Pancreatic cancer exosomes initiate pre-metastatic niche formation in the liver. Nat. Cell Biol. 17 (2015), doi: 10.1038/ncb3169.
3. H. Shao, J. Chung, L. Balaj, A. Charest, D. D. Bigner, B. S. Carter, F. H. Hochberg, X. O. Breakefield, R. Weissleder, H. Lee, Protein typing of circulating microvesicles allows real-time monitoring of glioblastoma therapy. Nat. Med. 18 (2012), doi: 10.1038/nm.2994.
4. M. Mathieu, L. Martin-Jaular, G. Lavieu, C. Théry, Specificities of secretion and uptake of exosomes and other extracellular vesicles for cell-to-cell communication. Nat. Cell Biol. 21 (2019),, doi: 10.1038/s41556-018-0250-9.
5. G. Chen, A. C. Huang, W. Zhang, G. Zhang, M. Wu, W. Xu, Z. Yu, J. Yang, B. Wang, H. Sun, H. Xia, Q. Man, W. Zhong, L. F. Antelo, B. Wu, X. Xiong, X. Liu, L. Guan, T. Li, S. Liu, R. Yang, Y. Lu, L. Dong, S. McGettigan, R. Somasundaram, R. Radhakrishnan, G. Mills, Y. Lu, J. Kim, Y. H. Chen, H. Dong, Y. Zhao, G. C. Karakousis, T. C. Mitchell, L. M. Schuchter, M. Herlyn, E. J. Wherry, X. Xu, W. Guo, Exosomal PD-L1 contributes to immunosuppression and is associated with anti-PD-1 response. Nature. 560 (2018), doi: 10.1038/s41586-018-0392-8.
6. Z. Yang, M. J. LaRiviere, J. Ko, J. E. Till, T. Christensen, S. S. Yee, T. A. Black, K. Tien, A. Lin, H. Shen, N. Bhagwat, D. Herman, A. Adallah, M. H. O′Hara, C. M. Vollmer, B. W. Katona, B. Z. Stanger, D. Issadore, E. L. Carpenter, A multianalyte panel consisting of extracellular vesicle miRNAs and mRNAs, cfDNA, and CA19-9 shows utility for diagnosis and staging of pancreatic ductal adenocarcinoma. Clin. Cancer Res. 26 (2020), doi: 10.1158/1078-0432.CCR-19-3313.
7. K. S. Yang, H. Im, S. Hong, I. Pergolini, A. F. Del Castillo, R. Wang, S. Clardy, C. H. Huang, C. Pille, S. Ferrone, R. Yang, C. M. Castro, H. Lee, C. F. Del Castillo, R. Weissleder, Multiparametric plasma EV profiling facilitates diagnosis of pancreatic malignancy. Sci. Transl. Med. (2017), doi: 10.1126/scitranslmed.aal3226.
8. A. Hoshino, H. S. Kim, L. Bojmar, K. E. Gyan, M. Cioffi, J. Hernandez, C. P. Zambirinis, G. Rodrigues, H. Molina, S. Heissel, M. T. Mark, L. Steiner, A. Benito-Martin, S. Lucotti, A. Di Giannatale, K. Offer, M. Nakajima, C. Williams, L. Nogués, F. A. Pelissier Vatter, A. Hashimoto, A. E. Davies, D. Freitas, C. M. Kenific, Y. Ararso, W. Buehring, P. Lauritzen, Y. Ogitani, K. Sugiura, N. Takahashi, M. Alečković, K. A. Bailey, J. S. Jolissant, H. Wang, A. Harris, L. M. Schaeffer, G. Garcia-Santos, Z. Posner, V. P. Balachandran, Y. Khakoo, G. P. Raju, A. Scherz, I. Sagi, R. Scherz-Shouval, Y. Yarden, M. Oren, M. Malladi, M. Petriccione, K. C. De Braganca, M. Donzelli, C. Fischer, S. Vitolano, G. P. Wright, L. Ganshaw, M. Marrano, A. Ahmed, J. DeStefano, E. Danzer, M. H. A. Roehrl, N. J. Lacayo, T. C. Vincent, M. R. Weiser, M. S. Brady, P. A. Meyers, L. H. Wexler, S. R. Ambati, A. J. Chou, E. K. Slotkin, S. Modak, S. S. Roberts, E. M. Basu, D. Diolaiti, B. A. Krantz, F. Cardoso, A. L. Simpson, M. Berger, C. M. Rudin, D. M. Simeone, M. Jain, C. M. Ghajar, S. K. Batra, B. Z. Stanger, J. Bui, K. A. Brown, V. K. Rajasekhar, J. H. Healey, M. de Sousa, K. Kramer, S. Sheth, J. Baisch, V. Pascual, T. E. Heaton, M. P. La Quaglia, D. J. Pisapia, R. Schwartz, H. Zhang, Y. Liu, A. Shukla, L. Blavier, Y. A. DeClerck, M. LaBarge, M. J. Bissell, T. C. Caffrey, P. M. Grandgenett, M. A. Hollingsworth, J. Bromberg, B. Costa-Silva, H. Peinado, Y. Kang, B. A. Garcia, E. M. O′Reilly, D. Kelsen, T. M. Trippett, D. R. Jones, I. R. Matei, W. R. Jarnagin, D. Lyden, Extracellular Vesicle and Particle Biomarkers Define Multiple Human Cancers. Cell. 182 (2020), doi: 10.1016/j.cell.2020.07.009.
9. K. Beard, D. F. Meaney, D. Issadore, Clinical Applications of Extracellular Vesicles in the Diagnosis and Treatment of Traumatic Brain Injury. J. Neurotrauma. 37 (2020),, doi: 10.1089/neu.2020.6990.
10. S. Muraoka, A. M. DeLeo, M. K. Sethi, K. Yukawa-Takamatsu, Z. Yang, J. Ko, J. D. Hogan, Z. Ruan, Y. You, Y. Wang, M. Medalla, S. Ikezu, M. Chen, W. Xia, S. Gorantla, H. E. Gendelman, D. Issadore, J. Zaia, T. Ikezu, Proteomic and biological profiling of extracellular vesicles from Alzheimer's disease human brain tissues. Alzheimer's Dement. 16 (2020), doi: 10.1002/alz.12089.
11. K. Beard, Z. Yang, M. Haber, M. Flamholz, R. Diaz-Arrastia, D. Sandsmark, D. F. Meaney, D. Issadore, Extracellular vesicles as distinct biomarker reservoirs for mild traumatic brain injury diagnosis. Brain Commun. 3 (2021), doi: 10.1093/braincomms/fcab151.
12. N. Habib, I. Avraham-Davidi, A. Basu, T. Burks, K. Shekhar, M. Hofree, S. R. Choudhury, F. Aguet, E. Gelfand, K. Ardlie, D. A. Weitz, O. Rozenblatt-Rosen, F. Zhang, A. Regev, Massively parallel single-nucleus RNA-seq with DroNc-seq. Nat. Methods. 14 (2017), doi: 10.1038/nmeth.4407.
13. A. M. Klein, L. Mazutis, I. Akartuna, N. Tallapragada, A. Veres, V. Li, L. Peshkin, D. A. Weitz, M. W. Kirschner, Droplet barcoding for single-cell transcriptomics applied to embryonic stem cells. Cell. 161 (2015), doi: 10.1016/j.cell.2015.04.044.
14. E. Z. Macosko, A. Basu, R. Satija, J. Nemesh, K. Shekhar, M. Goldman, I. Tirosh, A. R. Bialas, N. Kamitaki, E. M. Martersteck, J. J. Trombetta, D. A. Weitz, J. R. Sanes, A. K. Shalek, A. Regev, S. A. McCarroll, Highly parallel genome-wide expression profiling of individual cells using nanoliter droplets. Cell. 161 (2015), doi: 10.1016/j.cell.2015.05.002.
15. K. Grosselin, A. Durand, J. Marsolier, A. Poitou, E. Marangoni, F. Nemati, A. Dahmani, S. Lameiras, F. Reyal, O. Frenoy, Y. Pousse, M. Reichen, A. Woolfe, C. Brenan, A. D. Griffiths, C. Vallot, A. Gérard, High-throughput single-cell ChIP-seq identifies heterogeneity of chromatin states in breast cancer. Nat. Genet. 51 (2019), doi: 10.1038/s41588-019-0424-9.
16. A. E. Saliba, A. J. Westermann, S. A. Gorski, J. Vogel, Single-cell RNA-seq: Advances and future challenges. Nucleic Acids Res. 42 (2014),, doi: 10.1093/nar/gku555.
17. P. Beekman, A. Enciso-Martinez, H. S. Rho, S. P. Pujari, A. Lenferink, H. Zuilhof, L. W. M. M. Terstappen, C. Otto, S. Le Gac, Immuno-capture of extracellular vesicles for individual multi-modal characterization using AFM, SEM and Raman spectroscopy. Lab Chip. 19 (2019), doi: 10.1039/c9lc00081j.
18. K. Lee, K. Fraser, B. Ghaddar, K. Yang, E. Kim, L. Balaj, E. A. Chiocca, X. O. Breakefield, H. Lee, R. Weissleder, Multiplexed Profiling of Single Extracellular Vesicles. ACS Nano. 12 (2018), doi: 10.1021/acsnano.7b07060.
19. R. P. Carney, S. Hazari, M. Colquhoun, D. Tran, B. Hwang, M. S. Mulligan, J. D. Bryers, E. Girda, G. S. Leiserowitz, Z. J. Smith, K. S. Lam, Multispectral Optical Tweezers for Biochemical Fingerprinting of CD9-Positive Exosome Subpopulations. Anal. Chem. 89 (2017), doi: 10.1021/acs.analchem.7b00017.
20. Z. J. Smith, C. Lee, T. Rojalin, R. P. Carney, S. Hazari, A. Knudson, K. Lam, H. Saari, E. L. Ibañez, T. Viitala, T. Laaksonen, M. Yliperttula, S. Wachsmann-Hogiu, Single exosome study reveals subpopulations distributed among cell lines with variability related to membrane content. J. Extracell. Vesicles. 4 (2015), doi: 10.3402/jev.v4.28533.
21. K. B. Johnsen, J. M. Gudbergsson, T. L. Andresen, J. B. Simonsen, What is the blood concentration of extracellular vesicles? Implications for the use of extracellular vesicles as blood-borne biomarkers of cancer. Biochim. Biophys. Acta-Rev. Cancer. 1871 (2019),, doi: 10.1016/j.bbcan.2018.11.006.
22. J. Ko, Y. Wang, K. Sheng, D. A. Weitz, R. Weissleder, Sequencing-Based Protein Analysis of Single Extracellular Vesicles. ACS Nano. 15 (2021), doi: 10.1021/acsnano.1c00782.
23. C. Liu, X. Xu, B. Li, B. Situ, W. Pan, Y. Hu, T. An, S. Yao, L. Zheng, Single-Exosome-Counting Immunoassays for Cancer Diagnostics. Nano Lett. 18, 4226-4232 (2018).
24. P. Wei, F. Wu, B. Kang, X. Sun, F. Heskia, A. Pachot, J. Liang, D. Li, Plasma extracellular vesicles detected by Single Molecule array technology as a liquid biopsy for colorectal cancer. J. Extracell. Vesicles. 9 (2020), doi: 10.1080/20013078.2020.1809765.
25. F. Wu, Y. Gu, B. Kang, F. Heskia, A. Pachot, M. Bonneville, P. Wei, J. Liang, PD-L1 detection on circulating tumor-derived extracellular vesicles (T-EVs) from patients with lung cancer. Transl. Lung Cancer Res. 10 (2021), doi: 10.21037/tlcr-20-1277.
26. D. M. Rissin, C. W. Kan, T. G. Campbell, S. C. Howes, D. R. Fournier, L. Song, T. Piech, P. P. Patel, L. Chang, A. J. Rivnak, others, Single-molecule enzyme-linked immunosorbent assay detects serum proteins at subfemtomolar concentrations. Nat. Biotechnol. 28, 595-599 (2010).
27. L. Cohen, N. Cui, Y. Cai, P. M. Garden, X. Li, D. A. Weitz, D. R. Walt, Single Molecule Protein Detection with Attomolar Sensitivity Using Droplet Digital Enzyme-Linked Immunosorbent Assay. ACS Nano. 14 (2020), doi: 10.1021/acsnano.0c02378.
28. C. Wu, P. M. Garden, D. R. Walt, Ultrasensitive Detection of Attomolar Protein Concentrations by Dropcast Single Molecule Assays. J. Am. Chem. Soc. 142 (2020), doi: 10.1021/jacs.0c04331.
29. S. Cho, J. Yi, Y. Kwon, H. Kang, C. Han, J. Park, Multifluorescence Single Extracellular Vesicle Analysis by Time-Sequential Illumination and Tracking. ACS Nano (2021), doi: 10.1021/acsnano.1c02556.
30. V. Yelleswarapu, J. R. Buser, M. Haber, J. Baron, E. Inapuri, D. Issadore, Mobile platform for rapid sub-picogram-per-milliliter, multiplexed, digital droplet detection of proteins. Proc. Natl. Acad. Sci. U. S. A. 116 (2019), doi: 10.1073/pnas. 1814110116.
31. V. R. Yelleswarapu, H.-H. Jeong, S. Yadavali, D. Issadore, Ultra-high throughput detection (1 million droplets per second) of fluorescent droplets using a cell phone camera and time domain encoded optofluidics. Lab Chip. 17, 1083-1094 (2017).
32. C. W. Kan, C. I. Tobos, D. M. Rissin, A. D. Wiener, R. E. Meyer, D. M. Svancara, A. Comperchio, C. Warwick, R. Millington, N. Collier, D. C. Duffy, Digital enzyme-linked immunosorbent assays with sub-attomolar detection limits based on low numbers of capture beads combined with high efficiency bead analysis. Lab Chip. 20 (2020), doi: 10.1039/d0lc00267d.
33. L. Chang, D. M. Rissin, D. R. Fournier, T. Piech, P. P. Patel, D. H. Wilson, D. C. Duffy, Single molecule enzyme-linked immunosorbent assays: theoretical considerations. J. Immunol. Methods. 378, 102-115 (2012).
34. S. Dube, J. Qin, R. Ramakrishnan, Mathematical analysis of copy number variation in a DNA sample using digital PCR on a nanofluidic device. PLoS One. 3 (2008), doi: 10.1371/journal.pone.0002876.
35. H.-H. Jeong, V. R. Yelleswarapu, S. Yadavali, D. Issadore, D. Lee, Kilo-scale droplet generation in three-dimensional monolithic elastomer device (3D MED). Lab Chip. 15, 4387-4392 (2015).
36. F. J. MacWilliams, N. J. A. Sloane, Pseudo-random sequences and arrays. Proc. IEEE. 64, 1715-1729 (1976).
37. Y. Zhang, C. H. Pak, Y. Han, H. Ahlenius, Z. Zhang, S. Chanda, S. Marro, C. Patzke, C. Acuna, J. Covy, W. Xu, N. Yang, T. Danko, L. Chen, M. Wernig, T. C. Südhof, Rapid single-step induction of functional neurons from human pluripotent stem cells. Neuron. 78 (2013), doi: 10.1016/j.neuron.2013.05.029.
38. M. Sardar Sinha, A. Ansell-Schultz, L. Civitelli, C. Hildesjö, M. Larsson, L. Lannfelt, M. Ingelsson, M. Hallbeck, Alzheimer's disease pathology propagation by exosomes containing toxic amyloid-beta oligomers. Acta Neuropathol. 136 (2018), doi: 10.1007/s00401-018-1868-1.
39. C. Frühbeis, D. Fröhlich, W. P. Kuo, E. M. Krämer-Albers, Extracellular vesicles as mediators of neuron-glia communication. Front. Cell. Neurosci. (2013),, doi: 10.3389/fncel.2013.00182.
40. Y. Yang, A. Boza-Serrano, C. J. R. Dunning, B. H. Clausen, K. L. Lambertsen, T. Deierborg, Inflammation leads to distinct populations of extracellular vesicles from microglia. J. Neuroinflammation. 15 (2018), doi: 10.1186/s12974-018-1204-7.
41. C. C. Chen, L. Liu, F. Ma, C. W. Wong, X. E. Guo, J. V. Chacko, H. P. Farhoodi, S. X. Zhang, J. Zimak, A. Ségaliny, M. Riazifar, V. Pham, M. A. Digman, E. J. Pone, W. Zhao, Elucidation of Exosome Migration Across the Blood-Brain Barrier Model In Vitro. Cell. Mol. Bioeng. 9 (2016), doi: 10.1007/s12195-016-0458-3.
42. N. Koliha, Y. Wiencek, U. Heider, C. Jüngst, N. Kladt, S. Krauthäuser, I. C. D. Johnston, A. Bosio, A. Schauss, S. Wild, A novel multiplex bead-based platform highlights the diversity of extracellular vesicles. J. Extracell. Vesicles. 5 (2016), doi: 10.3402/jev.v5.29975.
43. S. Kuypers, N. Smisdom, I. Pintelon, J. P. Timmermans, M. Ameloot, L. Michiels, J. Hendrix, B. Hosseinkhani, Unsupervised Machine Learning-Based Clustering of Nanosized Fluorescent Extracellular Vesicles. Small. 17 (2021), doi: 10.1002/smll.202006786.
44. R. J. Fulton, R. L. McDade, P. L. Smith, L. J. Kienker, J. R. Kettman, in Clinical Chemistry (1997), vol. 43.
45. X. Wang, W. Zhong, J. Bu, Y. Li, R. Li, R. Nie, C. Xiao, K. Ma, X. Huang, Y. Li, Exosomal protein CD82 as a diagnostic biomarker for precision medicine for breast cancer. Mol. Carcinog. 58 (2019), doi: 10.1002/mc.22960.
46. Y. Yoshioka, N. Kosaka, Y. Konishi, H. Ohta, H. Okamoto, H. Sonoda, R. Nonaka, H. Yamamoto, H. Ishii, M. Mori, K. Furuta, T. Nakajima, H. Hayashi, H. Sugisaki, H. Higashimoto, T. Kato, F. Takeshita, T. Ochiya, Ultra-sensitive liquid biopsy of circulating extracellular vesicles using ExoScreen, 1-8 (2014).
47. Z. Chen, Q. Liang, H. Zeng, Q. Zhao, Z. Guo, R. Zhong, M. Xie, X. Cai, J. Su, Z. He, L. Zheng, K. Zhao, Exosomal CA125 as A promising biomarker for ovarian cancer diagnosis. J. Cancer. 11 (2020), doi: 10.7150/jca.48531.
48. P. Zhang, X. Wu, G. Gardashova, Y. Yang, Y. Zhang, L. Xu, Y. Zeng, Molecular and functional extracellular vesicle analysis using nanopatterned microchips monitors tumor progression and metastasis. Sci. Transl. Med. 12 (2020), doi: 10.1126/scitranslmed.aaz2878.
49. P. Zhang, X. Zhou, Y. Zeng, Multiplexed immunophenotyping of circulating exosomes on nano-engineered ExoProfile chip towards early diagnosis of cancer. Chem. Sci. 10 (2019), doi: 10.1039/c9sc00961b.
50. S. S. Adav, J. E. Park, S. K. Sze, Quantitative profiling brain proteomes revealed mitochondrial dysfunction in Alzheimer's disease. Mol. Brain. 12 (2019), doi: 10.1186/s13041-019-0430-y.
51. M. Imbeault, R. Lodge, M. Ouellet, M. J. Tremblay, Efficient magnetic bead-based separation of HIV-1-infected cells using an improved reporter virus system reveals that p53 up-regulation occurs exclusively in the virus-expressing cell population. Virology. 393 (2009), doi: 10.1016/j.virol.2009.07.009.
52. Y. Wang, V. Shah, A. Lu, E. Pachler, B. Cheng, D. Di Carlo, Counting of enzymatically amplified affinity reactions in hydrogel particle-templated drops. Lab Chip. 21 (2021), doi: 10.1039/d11c00344e.

### Methods

### Microfluidic Device Fabrication

DEVA incldues several components to enable high throughput droplet base single EV detection (FIG. 1C): (a) parallelized droplet generators that partition the mixture of bead and HRP substrate into picolitre sized droplets, (b) a delay line for enzymatic amplification reaction, and (c) the parallelized microfluidic channel where fluorescence signal is recorded. All microfluidic devices used in this work were fabricated using standard soft lithography. A thin layer of SU8 (MicroChem) was first spin coated on top of a silicon wafer. The spin rate was altered based on the targeted height. The SU8 later was then patterned via standard photolithography process: UV exposure, development, and baking. Afterwards, the wafer with patterned SU8 was silanized to complete the mold fabrication process. PDMS was well mixed with curing agent at a 10:1 ratio, poured onto the wafer with the SU8 mold, and degassed until there was no visible air bubbles. Then the PDMS was placed into a 65° C. over for at least 1 hour. The PDMS piece was eventually cut off from the mold. Holes for inlet and outlets were punched by a 1.5 mm disposable biopsy punch (INTERGRA). The assembly of the PDMS microfluidic device was conducted in the University of Pennsylvania's Nanofabrication facility, The Singh Center. First, conventional soft lithography was used to fabricate Si/SU-8 molds and PDMS replicates with microfluidic droplet generators. Also using conventional soft lithography, we fabricated Si/Su-8 molds and PDMS replicates that contain with microfluidic droplet generators and channels for droplet incubation and fluorescence detection. The design files are included separately. The two PDMS pieces and a glass substrate were subsequently treated with an oxygen plasma (Anatech SCE-106 Barrel Asher) at 100 W for 30 seconds, aligned, and bonded. Alignment was done manually under a stereoscope. To make the μfluidic channel hydrophobic, we treated the devices with 1% silane (Trichloro (1H,1H,2H,2H-perfluorooctyl) silane; Sigma) diluted in HFE 7500 Engineering Fluid (3M) for 1 hour and then flush the channel by HFE 7500 Engineering Fluid.

### Optical Setup

DEVA's detection device is placed in a laser-cut acrylic chip (⅛″) holder that aligns the chip under the RGB machine vision camera (Grasshopper3 USB3,GS3-U3-23S6C-C; FLIR). The chip is illuminated by blue and green lasers housed in an RGB laser module (λ_{exBlue}=457 nm, 3.66 W; λ_{exGreen}=528 nm, 1.25 W; Techhood), in which laser beams are guided by dichroic mirrors to exit through a single aperture. The collinear laser beams pass through a 20° circle tophat diffuser (ED1-C20-MD; Thorlabs) as they exit the module, creating a ˜20 mm diameter spotlight that illuminates the entire 15×9 mm² field of view uniformly. The emitted light from the detection device passes through a multi-bandpass filter (λ_{cwl}=485±10 nm, 559.5±12.5 nm, FF01-387/485/559/649-25; Semrock) and a C-mount macro zoom lens (MLM3X-MP; Computar) as it reaches the camera. The light sources are driven using a TTL module and a microcontroller (Arduino Mega2560) that modulates each laser according to its unique MLS pattern and triggers the camera to begin exposure in phase with the MLS. The MLS patterns were selected as described previously (1, 2). The videos of flowing droplets are taken using the FlyCap2 software (2.11.3.121; Point Grey Research) and stored locally for subsequent processing and analysis.

### Human Neuron Cell Culture

Human iPS cells were differentiated into neurons using an established protocol (3). Briefly, iPS cells were infected with two lentiviral vectors: TetO-mNgn2-T2A-PuroR and Ubiq-rTTA. Neuronal differentiation was initiated by exposure to 2 ug/ml doxycycline (Sigma), followed 24 hrs later by 5 ug/ml puromycin (Sigma) selection for cells that possessed these two lentiviral vectors. Differentiating cells were then plated on a deformable silicone membrane (0.002-in. thickness, Specialty Manufacturing) at a density of 60,000 cells/ml, that was precoated with Matrigel (Sigma) (4). Cells were grown in Neurobasal-A medium (Gibco) with B27 (Life Technologies), glutamax (Life technologies), 5 mM glucose, 10 mM sodium pyruvate, 10 ng/ml NT-3 (Peprotech), 10 ng/ml Brain-derived neurotrophic factor (Peprotech), and 2 μg/ml doxycycline. Differentiated neurons were cultured for 18 days. Doxycycline was discontinued on day 10. Media was changed every 3 or 4 days where it was collected and stored at −20° C.

### Preparation of Antibody Coated Magnetic Microbeads and Detection Antibody Biotinylation

Capture antibody (anti-human CD81 antibody, 130-124-538, Miltenyi) was coated on the surface of 5.4 μm Fluorescent Yellow Carboxyl Magnetic Particles (FCM-5052-2, Spherotech) following the protocol from PolyLink Protein Coupling Kit (24350-1, Polyscience). Briefly, we activated the carboxyl groups on the microbeads by water soluble carbodiimide (EDAC). The carbodiimide reacts with the carboxyl group and creates an active ester that is reactive towards primary amines on the capture antibody.

Detection antibodies were biotinylated following the protocol of one-step antibody biotinylation kit (Miltenyi). Briefly, anti-human CD81 antibody (130-124-538, Miltenyi) was prepared at the concentration of 100 μg/mL in PBS. 100 μL of CD81 detection antibody was added into one Miltenyi well containing lyophilized powder. The solution was suspended repeatedly to mix the lyophilized powder thoroughly. The mixture was incubated at room temperature for 24 hours before usage.

### Video Processing and Analysis

This droplet detection workflow relies on modulating the excitation sources with a pseudorandom sequence at a rate greater than the frame rate of the camera, which enables the moving fluorescent beads and droplets to be imaged as patterned streaks. Correlating the fluorescence signal of the patterned droplet with the expected sequence results in a distinct peak and enables an individual droplet to be resolved amongst neighboring droplets, with a minimum separation of 3.5 times the droplet diameter (2). Simulation experiments and subsequent receiver operator characteristic (ROC) analysis, in which the number of droplets per channel is gradually increased from N=1 to N=27, reveal that as many as 20 patterned droplets can be detected in a single channel with an area under the curve (AUC)>0.95 (2). The length of the droplet streak Lₛₜᵣₑₐₖ is set to be ⅓ of the channel length according to the equation Lₛₜᵣₑₐₖ=v×Tₑₓₚ, where v is the velocity of the droplet and Tₑₓₚ is the exposure time of the camera. As such, for a given velocity v, the optimal exposure time is determined to image the droplet at least twice as it moves (2).

Based on this mechanism described above. The acquired videos were processed with a custom workflow developed in MATLAB 2021a (Mathworks, Inc.) and computationally accelerated with a graphical processing unit (Nvidia GeForce ABC) to a) identify droplets containing microbeads, and b) determine if the substrate encapsulated within the droplet was enzymatically activated (FIG. 3). First, each frame of the collected RGB videos was separated into red and blue color components, corrected for optical aberrations, and segmented by fluidic channel. Each segmented channel was then converted into a 1D intensity profile, Sᵢ^{B,R}, where i is the index for the channel and B corresponds to the blue component of the image and R the red component of the image, by subtracting the frame background and integrating along the cross-section of the channel. Finally, the presence of beads and droplets within a channel was determined by analyzing the peaks in the cross correlation of Sᵢ^{B,R} with the corresponding MLS pattern scaled to match the streak length of the beads and droplets in each frame. Proper scaling of the MLS pattern for a channel, equivalent to identifying the velocity within that channel, was accomplished by finding the maximum of the cross correlation between Sᵢ^{B,R} and the corresponding MLS patterns when scaled for streak lengths ranging from 1.6 mm to 9.6 mm. The subsequent peak detection algorithm was modified based on the Matlab findpeaks function. The thresholds for peak detection in the bead channel and the substrate channel were determined by flowing a) control samples without beads and b) control samples containing beads but not EVs respectively. A peak in the blue channel but not the red channel indicated a droplet with a bead without an EV, and a peak in both the blue and red channels indicated a positive droplet, i.e. a bead bound to a single EV. These quantities are stored for downstream AEVB calculations.

### 10-Channel Droplet Microfluidics Device

A droplet microfluidic device with 10 parallel flow-focusing generators is fabricated using “double-sided imprinting” method, as previously reported (5). The microfluidic device contains four 200 μm deep delivery channels, through which the fluids are distributed to individual droplet generators. The droplet generators are connected to the delivery channels by through-PDMS vias, with a depth of 150 μm. The flow-focusing generators are designed to be 30 μm deep, matching the dimension of the flow-focusing nozzle. The channel dimensions are designed to satisfy the design criteria:

$$2 N ( \frac{{R}_{d}}{{R}_{dev}} ) < 0.01 ,$$

where N is the number of the parallel devices, R_{d} and R_{dev} the fluidic resistance of the delivery channel and the device. respectively. The fluidic resistance of the channel can be calculated from

$$R = 12 ( \frac{μ L}{{wh}^{3}} ) {( 1 - 0.63 ( \frac{h}{w} ) )}^{- 1} ,$$

where μ is the viscosity of the fluid, and w,h,L are the width, height, and length of the channel, respectively. The design criteria ensure even fluid distribution throughout the devices since the fluidic resistance in the delivery channel is negligible, compared to that of the device channel.

### Calculation of Partitioning Error

If we define m as the number of targets in sample, n as the total number of partitions, λ as the average number of targets per partition. Then we have

$$λ = \frac{m}{n}$$

If we define the percentage of empty partitions (E), based on binominal distribution (6) we could get

$$λ = - ln ( E )$$

Partitioning errors occur as targets may distribute differently among partitions from one experiment to the next. In a set of experiments, the number of empty partitions E would have variance that propagates to a corresponding variance in the calculated concentration λ (6). The partition error

$${u}_{p} = \frac{Δ m}{m} = \frac{{λ}_{max} - {λ}_{min}}{λ} = \frac{n ( {λ}_{max} - {λ}_{min} )}{n λ} .$$

Based on the model from Dube et al (7),

$${λ}_{min} = - ln ( E - {Z}_{C} \sqrt{\frac{E ( 1 - E )}{n}} ) .$$

while

$${λ}_{max} = - ln ( E - {Z}_{C} \sqrt{\frac{E ( 1 - E )}{n}} ) .$$

If we consider

$${σ}_{E} = \sqrt{\frac{E ( 1 - E )}{n} )} .$$

### Then we get

$${u}_{p} = \frac{Δ m}{m} = \frac{{λ}_{max} - {λ}_{min}}{λ} = \frac{n ( {λ}_{max} - {λ}_{min} )}{n λ} = \frac{1}{λ} ln ( \frac{E + {Z}_{c {σ}_{E}}}{E + {Z}_{c {σ}_{E}}} )$$

TABLE 1

| Assay | | LOD | | Number | |
|---|---|---|---|---|---|
| Target | Platform | (μL⁻¹) | AEVB_{b} | of beads | Reference |
| iFN | SIMOA, | 210 | 1% | 25,000 | (8) |
| | Quanterix | | | | |
| CD9-CD63 | SIMOA, | 34 | 0.6% | 25,000 | (9) |
| EVs | Quanterix | | | | |
| iFN | Customized | 18 | 0.2-1% | 60,000 | (8) |
| | microarray | | | | |
| CD63-GPC1 | Customized | 10 | Did not | 45,000 | (10) |
| EVs | microarray | | report | | |
| CD81-CD81 | DEVA, μMD | 9 | 0.023% | 1,000,000 | This work |

Comparing technical features of DEVA with commercial or research-based platforms to perform digital ELISA assays. Benefiting from its high throughput, DEVA can analyze a larger number of beads and achieves a low background level (AEVBb).

TABLE 2

| | | | Number of single | |
|---|---|---|---|---|
| | Number of | | EVs analyzed | |
| Method | compartments | | per assay | Reference |
| ddPCR | N/A | | 1,100 | (11) |
| dELISA, | 25,000 | microwells | 2,500 | (8, 12) |
| SIMOA | | | | |
| dELISA, | 20,000 | microwells | 4,500-10,000 | (10, 13) |
| customized | 1,000,000 | droplets | | |
| microarray | | | | |
| Droplet | 10,000,000 | droplets | 100,000 | This work |
| based | | | | |
| dELISA | | | | |

Comparing DEVA and typical commercial or research-based single EV analysis platforms that used digital assays. Based on DEVA's high throughput, more single EVs could be analyzed in one assay.

### Measuring Human Plasma EV Using DEVA

We tested the feasibility of detecting endogenous EVs in a human plasma sample using DEVA (FIG. 16). Due to the presence of endogenous CD81+/CD81+ EVs in human plasma, we cannot do spike in experiments of known concentrations of target EVs into a background that contains no target EVs. Instead, we have validated the feasibility of detecting EVs in plasma by quantifying the number of CD81⁺/CD81⁺ EVs in human plasma at various dilutions. To validate the specificity of DEVA in human plasma, we compared these results to a negative control in which we replace the CD81 capture and label antibodies with an isotype antibody control (human IgG1, Miltenyi Biotec). In our measurements on DEVA, the isotype control showed consistent background at AEVB=0.134% (CV=15%) over three orders or magnitude of human plasma EV spiked-in the experiment.

### Figures

FIG. 1. High throughput droplet based extracellular vesicle assay (DEVA). (A) Detecting specific EVs at single particle level is challenging, as there exists enormous EV background (108) in just 10 μL of human plasma. (B) Bead based digital ELISA assay for single specific EV detection. i. Paramagentic, fluorescent beads were coated with capture antibody. ii. Antibody-coated beads are added to a sample containing target and background EVs. iii. After Bead-EV incubation, unbounded EVs are washed away. iv. Biotinylated detection antibody is added to label the captured EVs. v. Streptavidin-HRP is then added and bond with biotinylated detection antibody to form an enzyme-labeled immunocomplex. vi. The beads are mixed with HRP substrate and then be partitioned into droplets. Droplets that contain one immunocomplex become fluorescent due to enzyme-substrate reaction. (C) i. Miniaturized microfluidic platform for DEVA that integrates the droplet generation section, incubation section, and detection section. ii. All droplets flow through a delay line chamber for incubation. Afterwards, all droplets flow through a detection section consisting of 90 parallelized microfluidic channels. iii. Based on Poisson statistics, droplet number should be 10 times the bead number to ensure that droplets have at most one bead. A parallelized flow focusing droplet generator was developed to meet this requirement. iv. The EV-bead immunocomplex turns the droplet fluorescent during the incubation in the delay line. v. A machine vision camera records the encoded fluorescent signal of 90 parallelized microfluidic channels and transfers the video to cloud computation for image analysis. S is short for substrate while B is short for beads. S+|B+ means a droplet expresses signal from both substrate and bead. S−|B+ means a droplet only contains bead signal. S−|B− means blank droplet.

FIG. 2. Theoretical calculation demonstrating the benefits of more beads in DEVA. (A) When background level is low, analyzing more beads is essential to minimize the imprecision coming from Poisson noise and to achieve a low limit of detection (LOD). Inlet shows the schematic of LOD in a typical digital assay. LOD is calculated based on mean background level (AEVBb) plus three times the standard deviation. (B) Shows the number of beads that can sufficiently minimize Poisson noise (1 dB) at different background levels. (C) Shows relative partition error (ϵ_p, the ratio of error over readout) will impair signal readout when there exists multiple target analyte per microbead (λ>1). Different bead numbers are indicated by different colors: purple 10 k; brown 30 k; red 100 k; navy 300 k; magenta 1M. (D) More beads enable a higher limit of linearity (LOL) by keeping the partition error less than 10% of output signal. Upper left inlet shows the schematic of limit of linearity at high EV concentrations.

FIG. 3. High throughput detection schematic for DEVA. (A) A schematic revealing the DEVA detection section. A microcontroller synchronizes the laser excitation and the camera recording. A diffuser is used to ensure uniform excitation across the chip. Multipass filters between the detection chip and the camera reduces background signal. The video is transmitted to cloud that uses parallelized computation for our intensive image processing. (B) The fluorescent signal from the substrate and bead will be encoded by different lasers respectively. Truth table showing the interpretation on the fluorescent signal. S, substrate. B, bead. (C) Demonstration of a real frame recorded by the camera. (D) Decoding fluorescent signal in the bead and substrate channels respectively. The 3D surface function demonstrates the high correlation signal for bead or substrate identification during image process. (E) Example figures show we can identify signal from a bead only (left) and a bead that has captured a single EV (right). Schematics and real camera frames are shown respectively, followed by the correlation signal in the bead channel as well as the substrate channel.

FIG. 4. Characterization of human neuron EVs for DEVA. (A) SEM shows an EV being captured on antibody coated microbeads. Scale bar shows 300 nm. (B) NTA analysis shows the size distribution as well as the concentration of human neuron derived EVs. (C) i. Schematic of Nanoview to reveal EV surface protein profiling by immunocapture and immunolabeling. Red stands for CD63, Green stands for CD81, Blue stands for CD9. ii. Fluorescent image recorded by Nanoview showing the surface protein profiling on captured EVs. iii. Nanoview chip captured most human neuron EVs on the anti-CD81-coated chip. * indicates p<0.05, *** indicates p<0.001. iv. 11% of human neuron EVs express at least one CD81 protein based on immunoisolation and protein calibration. v. Surface protein profiling of CD81+EVs revealed by Nanoview. Each marker group represents a distinct EV subpopulation with no overlap. vi. Among the CD81+EVs, 67% of them expressed at least one other CD81 protein.

### FIG. 5. Benchmarking and Characterization of DEVA.

(A) Fluorescent microscopic images showing DEVA assay on serial diluted human neuron EVs. (B) Calibration curve for DEVA detecting CD81/CD81 human neuron EVs spiked into PBS. Inset shows the detailed characterization. Error bars were calculated from experimental replicates (N=3). (C) Signal over background for the calibration curve in A. Inlet shows the zoomed-in view of signal-over-background (y-axis) from 0 to 4000 EVs/(x-axis). (D) Analyzing more beads improves DEVA LOD by minimizing the background imprecision coming from Poisson noise. Inlet shows that increasing the number of beads analyzed decreases the variance of the AEVBb measurement. Although the background level remains similar, a higher background variance impairs the LOD of DEVA. (E) Comparing the sensitivity and the dynamic range of DEVA, NTA, and Nanoview. The dynamic range of DEVA was calculated based on expectation. (F) Calibration curve for DEVA detecting CD81/CD81 human neuron EVs with background (bcg) EVs (background EV concentration in final sample volume is indicated by grey bar). Inlet shows detailed characterization. Error bars were calculated from experimental replicates (N=3). (G) Signal over background for the calibration curve for DEVA in complex media. Inlet shows the zoomed-in view from 0 to 5000 EVs/μL. (H) DEVA shows no significant difference (student t test) on the background level (AEVBb) in PBS or complex media.

### FIG. 6. A Photograph of the Experimental Setup of our DEVA Platform

### FIG. 7. Excitation Laser Characterization for DEVA

A blue laser (λexBlue=457 nm) was used to excite the signal from fluorescent microbeads while a green laser (λexGreen=528 nm) was used to excite the signal from fluorescent enzyme substrate.

### FIG. 8. DEVA Fluorescence Characterization.

A. DEVA quantification of spiked-in beads, showing excellent linearity (R²=0.989) over a large dynamic range (10²-10⁷). B. Comparison of the fluorescence intensity between droplets with beads versus those without (N=41). Welch's t-test was used to calculate the significance. C. Histogram showing the fluorescence intensity of droplets with and without fluorescent beads (N=41). D. Comparison of the fluorescence intensity between droplets with enzyme versus those without (N=118). Welch's t-test was used to calculate the significance. E. Histogram showing fluorescence intensity of droplets with and without enzyme (N=118).

### FIG. 9. High Throughput Droplet Generation Enabled by Parallelized Flow Focusing Generators.

Micrograph of a parallelized droplet generators in a ladder architecture fabricated in PDMS. Scale bar: 500 μm. Navy box indicates the single droplet generator shown in B. (B) A micrograph of a single droplet generator making water in oil emulsions. Scale bar: 200 μm. (C) A micrograph of the droplets generated by the ten parallelized droplet generators. Scale bar: 50 μm. (D) A histogram of the diameter of the droplets collected from the parallelized droplet generation device. The flow rate for the combined aqueous phase is 4.6 mL/hr and for the continuous flow rate of 60 mL/hr.

### FIG. 10. SEM Images of DEVA.

### SEM Images Showing Immuno-Captured Single EV on Bead

FIG. 11. Schematics on Determining the Ratio of CD81+EVs Among the total human neuron EVs using Qubit protein assays.

Isolated human neuron derived EVs were first lysed using 1% SDS buffer to measure the total EV protein cargo. Meanwhile, anti-CD81-coated beads were incubated with the same isolated human EVs to capture CD81⁺ EVs. After isolation and washing, CD81⁺ EVs that were captured on beads were lysed and measured using the same condition. After comparison, captured CD81⁺ EVs consist 11% of the protein cargo of human neuron derived EVs.

### FIG. 12. DEVA Optimization on Reagent Concentration.

A. Several concentrations of HRP enzyme and detection antibody were evaluated for DEVA assay optimization. Each assay condition was applied to an EV-inputted and a blank sample, and a resulting SNR was calculated based on the number of positive droplets detected in the EV-inputted versus the blank sample. B. At 1 nM HRP concentration, several detection antibody concentrations were added into blank samples without EVs to minimize unspecific binding. Error bars indicate standard deviation from replicates.

### FIG. 13. Supplementary Fluorescent Microscopic Images.

A. Additional fluorescent microscopic images showing the DEVA assay on serially diluted human neuron EVs. Each experiment had over 30 images analyzed for AEVB calculation. B. Quantitative analysis of AEVB from fluorescent microscope images.

FIG. 14. Human neuron EVs characterization on conventional tools. A. Isolated human neuron EVs characterized on Nanoparticle tracking analysis (NTA). B. Isolated human neuron EVs characterized on Nanoview platform. Error bars indicate standard deviation on both figures.

### FIG. 15. NTA Analysis on Isolated EVs.

A. NTA characterization on FBS EVs. B. NTA characterization on

healthy human plasma EVs. EV number was calculated based on NTA result then spiked- in DEVA experiments.

FIG. 16. Feasibility of measuring endogenous EVs in human plasma using DEVA. A. Schematic of the measurements carried out on Nanoview to quantify the surface proteins expressed on human plasma EV by immunocapture and immunolabeling. r stands for CD63, g stands for CD81, b stands for CD9. Nanoview chip was coated by anti-CD81 antibody. B. We found that 15% of human plasma EVs express at least one CD81 protein based on immunoisolation and protein calibration. C. Surface protein profiling of CD81⁺ EVs in human plasma by Nanoview. Among the CD81⁺ EVs, 8% of them expressed at least one other CD81 protein. D. A titration curve for DEVA when endogenous human CD81/CD81 EVs are measured at various dilutions of 20 μL of human plasma into 100 μL sample volume. The x axis was estimated using NTA to measure human plasma (1.2×10¹²/mL), of which we assumed 1% were EVs based on prior literature (14). Our estimate of 1.2×10¹⁰ EVs/mL agrees with what has been previously reported in literature (14, 15). Error bars indicate standard deviations from replicates. Some error bars are too small to be seen on the figure.

### Aspects

The following Aspects are illustrative only and do not limit the scope of the present disclosure or the appended claims. Any part or parts of any one or more Aspects can be combined with any part or parts of any one or more other Aspects.

Aspect 1. A method, comprising: contacting (i) a sample comprising a plurality of extracellular vesicles (EVs) and (ii) a detectable capture modality complementary to a target EV so as to form a first product; contacting the first product with a detection modality that associates with the target EV so as to form a second product that comprises at least one—and can comprise a plurality of—detectable capture modalities; and within a plurality of droplets, contacting the second product and a substrate that is reactive with the detection modality to produce a detectable signal indicative of the presence of the second product within the droplet; and optically interrogating the plurality of droplets.

As shown elsewhere herein (e.g., FIG. 1), a detectable capture modality can be a bead (which bead can be attractable by a magnetic field), which bead can also be associated with an antibody—which can be termed a capture antibody—that is complementary to an EV of interest. A detection modality can be (e.g., FIG. 1) an antibody that is complementary to an EV of interest. The detection modality—which can itself be an antibody—can also be associated with an enzyme, which enzyme can react with a substrate within a droplet and evolve a detectable signal, as shown in FIG. 1.

Aspect 2. The method of Aspect 1, wherein the second product is loaded into the plurality of droplets, the droplets having the substrate therein.

Aspect 3. The method of any one of Aspects 1-2, wherein the detectable capture modality comprises a bead associated with an antibody complementary to a first protein of the target EV, the bead optionally being a paramagnetic bead and/or a bead having a fluorescent signal. Example proteins include, without limitation, proteins which are found on exosomes, such as CD9, CF61, CD83. Other proteins can be, e.g., tissue specific proteins, such as EpCAM or EGFR for tumor tissue, NCAM1 for neurons, or GLAST for astrocytes.

Aspect 4. The method of any one of Aspects 1-3, wherein the detection modality comprises an antibody complementary to a protein of the target EV.

Aspect 5. The method of any one of Aspects 1-4, wherein the detection modality comprises an enzyme that reacts with detection modality, and wherein the signal is a color or a fluorescence.

Aspect 6. The method of any one of Aspects 1-5, wherein the method has a limit of detection (LOD) of about 9 EV/μL.

Aspect 7. The method of any one of Aspects 1-6, wherein a number of droplets is about 10 times a number of capture modalities, the number of droplets optionally being at least 10 times a number of capture modalities.

Aspect 8. The method of any one of Aspects 1-6, wherein a number of droplets is about 20 times the number of capture modalities.

Aspect 9. The method of any one of Aspects 1-8, further comprising: communicating the plurality of droplets through at least one channel of a microfluidic device; illuminating the droplets with a first time-domain modulated sequence of flashes from a first light source.

Aspect 10. The method of Aspect 9, wherein the first light source is configured to evolve the detectable signal indicative of the presence of the second product within the droplet.

Aspect 11. The method according to Aspect 9, wherein the first time-domain modulated sequence is a pseudorandom sequence.

Aspect 12. The method according to Aspect 9, wherein the first time-domain modulated sequence is a minimally correlating maximum length sequence

Aspect 13. The method according to Aspect 12, wherein the maximum length sequence comprises a beginning sequence and an end sequence and wherein the beginning sequence differs from the end sequence.

Aspect 14. The method according to Aspect 13, wherein the beginning sequence differs from the end sequence by at least 10%.

Aspect 15. The method according to Aspect 14, wherein the beginning sequence differs from the end sequence by at least 10%, and the middle sequence differs from the beginning and end sequences by at least 10%.

Aspect 16. The method according to Aspect 13, wherein the maximum length sequence further comprises a middle sequence, and the middle sequence differs from the beginning sequence and the end sequence.

Aspect 17. The method according to Aspect 12, wherein the maximum length sequence is 1/30 sec or less.

Aspect 18. The method according to Aspect 17, wherein the maximum length sequence is 1/60 sec or less.

Aspect 19. The method according to any one of Aspects 9-18, further comprising illuminating the droplets with a second time-domain modulated sequence of flashes from a second light source, the second time-domain modulated sequence differing from the first time-domain modulated sequence, the method further optionally comprising illuminating the droplets with a third time-domain modulated sequence of flashes from a third light source, the third time-domain modulated sequence differing from the first time-domain modulated sequence and the second time-domain modulated sequence.

Aspect 20. The method of Aspect 19, wherein the second light source is configured to evolve the detectable signal indicative of the presence of the detectable capture moiety but not the second product within the droplet.

Aspect 21. The method according to any one of Aspects 1-20, further comprising capturing a plurality of images of the droplets.

Aspect 22. The method according to Aspect 21, further comprising correlating an illumination pattern in the plurality of images to (1) the presence of absence of a detectable capture moiety (e.g., a detectable capture moiety that is not bound to a target EV) in a droplet and (2) the presence or absence of the second product (for example, a detectable capture moiety that is bound to a target EV that then effects a color change or other detectable signal in the droplet) in the droplet.

Aspect 23. The method of Aspect 22, further comprising correlating the presence or absence of the second product (e.g., a bead bound to target EV that then causes color change or other detectable signal within the droplet) to a condition of a source of the sample. Such a condition can be, e.g., a disease state, such as a cancer state.

Aspect 24. The method of Aspect 23, wherein the condition is a disease state.

Aspect 25. A system, the system being configured to perform the method of any one of Aspects 1-24.

Aspect 26. A system, comprising: a droplet generation section configured to form a plurality of droplets having therein a substrate and a final product comprising an EV and reactive with the substrate to produce a detectable signal indicative of the presence of the final product within the droplet; an incubation section in fluid communication with the droplet generation section and configured to communicate therein the droplets, the incubation section operable to provide a residence time for the droplets sufficient to give rise to the detectable signal indicative of the presence of the final product within the droplet; and a detection section, the detection section configured to communicate therein the plurality of droplets through at least one channel of a microfluidic device, illuminate the droplets with a first time-domain modulated sequence of flashes from a first light source, and capture a plurality of images of the droplets.

Aspect 27. The system of Aspect 26, wherein the detection section is configured to illuminate the droplets with a second time-domain modulated sequence of flashes from a second light source.

Aspect 28. The system of any one of Aspects 26-27, the system further configured to correlate an illumination pattern in the plurality of images with an expected pattern based at least on the first time domain modulated sequence to determine a number and/or position of droplets containing the final product.

Aspect 29. The system of any one of Aspects 26-28, further comprising a treatment section, the treatment section configured to contact (i) a sample comprising a plurality of extracellular vesicles (EVs) and (ii) a detectable capture modality complementary to a target EV so as to form a first product;

Aspect 30. The system of Aspect 29, wherein the treatment section is further configured to contact the pre-product with a detection modality that associates with the target EV so as to form the final product.

### REFERENCES

1. V. Yelleswarapu, J. R. Buser, M. Haber, J. Baron, E. Inapuri, D. Issadore, Mobile platform for rapid sub-picogram-per-milliliter, multiplexed, digital droplet detection of proteins. Proc. Natl. Acad. Sci. U.S.A. 116 (2019), doi: 10.1073/pnas. 1814110116.
2. V. R. Yelleswarapu, H.-H. Jeong, S. Yadavali, D. Issadore, Ultra-high throughput detection (1 million droplets per second) of fluorescent droplets using a cell phone camera and time domain encoded optofluidics. Lab Chip. 17, 1083-1094 (2017).
3. Y. Zhang, C. H. Pak, Y. Han, H. Ahlenius, Z. Zhang, S. Chanda, S. Marro, C. Patzke, C. Acuna, J. Covy, W. Xu, N. Yang, T. Danko, L. Chen, M. Wernig, T. C. Südhof, Rapid single-step induction of functional neurons from human pluripotent stem cells. Neuron. 78 (2013), doi: 10.1016/j.neuron.2013.05.029.
4. J. P. Dollé, A. Jaye, S. A. Anderson, H. Ahmadzadeh, V. B. Shenoy, D. H. Smith, Newfound sex differences in axonal structure underlie differential outcomes from in vitro traumatic axonal injury. Exp. Neurol. 300 (2018), doi: 10.1016/j.expneurol.2017.11.001.
5. H. H. Jeong, S. Yadavali, D. Issadore, D. Lee, Liter-scale production of uniform gas bubbles: Via parallelization of flow-focusing generators. Lab Chip. 17 (2017), doi: 10.1039/c71c00295e.
6. F. Bizouarn, Introduction to digital PCR. Methods Mol. Biol. 1160 (2014), doi: 10.1007/978-1-4939-0733-5_4.
7. S. Dube, J. Qin, R. Ramakrishnan, Mathematical analysis of copy number variation in a DNA sample using digital PCR on a nanofluidic device. PLoS One. 3 (2008), doi: 10.1371/journal.pone.0002876.
8. L. Cohen, N. Cui, Y. Cai, P. M. Garden, X. Li, D. A. Weitz, D. R. Walt, Single Molecule Protein Detection with Attomolar Sensitivity Using Droplet Digital Enzyme-Linked Immunosorbent Assay. ACS Nano. 14 (2020), doi: 10.1021/acsnano.0c02378.
9. P. Wei, F. Wu, B. Kang, X. Sun, F. Heskia, A. Pachot, J. Liang, D. Li, Plasma extracellular vesicles detected by Single Molecule array technology as a liquid biopsy for colorectal cancer. J. Extracell. Vesicles. 9 (2020), doi: 10.1080/20013078.2020.1809765.
10. C. Liu, X. Xu, B. Li, B. Situ, W. Pan, Y. Hu, T. An, S. Yao, L. Zheng, Single-Exosome-Counting Immunoassays for Cancer Diagnostics. Nano Lett. 18, 4226-4232 (2018).
11. J. Ko, Y. Wang, K. Sheng, D. A. Weitz, R. Weissleder, Sequencing- Based Protein Analysis of Single Extracellular Vesicles. ACS Nano. 15 (2021), doi: 10.1021/acsnano.1c00782.
12. F. Wu, Y. Gu, B. Kang, F. Heskia, A. Pachot, M. Bonneville, P. Wei, J. Liang, PD-L1 detection on circulating tumor-derived extracellular vesicles (T- EVs) from patients with lung cancer. Transl. Lung Cancer Res. 10 (2021), doi: 10.21037/tlcr-20-1277.
13. Q. Tian, C. He, G. Liu, Y. Zhao, L. Hui, Y. Mu, R. Tang, Y. Luo, S. Zheng, B. Wang, Nanoparticle Counting by Microscopic Digital Detection: Selective Quantitative Analysis of Exosomes via Surface-Anchored Nucleic Acid Amplification. Anal. Chem. 90 (2018), doi: 10.1021/acs.analchem.8b00189.
14. K. B. Johnsen, J. M. Gudbergsson, T. L. Andresen, J. B. Simonsen, What is the blood concentration of extracellular vesicles? Implications for the use of extracellular vesicles as blood-borne biomarkers of cancer. Biochim. Biophys. Acta-Rev. Cancer. 1871 (2019),, doi: 10.1016/j.bbcan.2018.11.006.
15. S. Jamaly, C. Ramberg, R. Olsen, N. Latysheva, P. Webster, T. Sovershaev, S. K. Brækkan, J. B. Hansen, Impact of preanalytical conditions on plasma concentration and size distribution of extracellular vesicles using Nanoparticle Tracking Analysis. Sci. Rep. 8 (2018), doi: 10.1038/s41598-018-35401-8.

1. A method, comprising:
contacting (i) a sample comprising a plurality of extracellular vesicles (EVs) and (ii) a detectable capture modality complementary to a target EV so as to form a first product:
contacting the first product with a detection modality that associates with the target EV so as to form a second product that comprises at least one detectable capture modalities; and
within a plurality of droplets, contacting the second product and a substrate that is reactive with the detection modality to produce a detectable signal indicative of the presence of the second product within the droplet; and
optically interrogating the plurality of droplets.

2. The method of claim 1, wherein the second product is loaded into the plurality of droplets, the droplets having the substrate therein.

3. The method of claim 1, wherein the detectable capture modality comprises a bead associated with an antibody complementary to a first protein of the target EV, the bead optionally being a paramagnetic bead and/or a bead having a fluorescent signal.

4. The method of claim 1, wherein the detection modality comprises an antibody complementary to a protein of the target EV.

5. The method of claim 1, wherein the detection modality comprises an enzyme that reacts with detection modality, and wherein the signal is a color or a fluorescence.

6. The method of claim 1, wherein the method has a limit of detection (LOD) of about 9 EV/μL.

7. The method of claim 1, wherein a number of droplets is about 10 times a number of capture modalities, the number of droplets optionally being at least 10 times a number of capture modalities.

8. The method of claim 1, wherein a number of droplets is about 20 times the number of capture modalities.

9. The method of claim 1, further comprising:
communicating the plurality of droplets through at least one channel of a microfluidic device:
illuminating the droplets with a first time-domain modulated sequence of flashes from a first light source.

10. The method of claim 9, wherein the first light source is configured to evolve the detectable signal indicative of the presence of the second product within the droplet.

11. The method according to claim 9, wherein the first time-domain modulated sequence is a pseudorandom sequence.

12. The method according to claim 9, wherein the first time-domain modulated sequence is a minimally correlating maximum length sequence

13. The method according to claim 12, wherein the maximum length sequence comprises a beginning sequence and an end sequence and wherein the beginning sequence differs from the end sequence.

14. The method according to claim 13, wherein the beginning sequence differs from the end sequence by at least 10%.

15. The method according to claim 14, wherein the beginning sequence differs from the end sequence by at least 10%, and the middle sequence differs from the beginning and end sequences by at least 10%.

16. The method according to claim 13, wherein the maximum length sequence further comprises a middle sequence, and the middle sequence differs from the beginning sequence and the end sequence.

17. The method according to claim 12, wherein the maximum length sequence is 1/30 sec or less.

18. The method according to claim 17, wherein the maximum length sequence is 1/60 sec or less.

19. The method according to claim 9, further comprising illuminating the droplets with a second time-domain modulated sequence of flashes from a second light source, the second time-domain modulated sequence differing from the first time-domain modulated sequence, the method further optionally comprising illuminating the droplets with a third time-domain modulated sequence of flashes from a third light source, the third time-domain modulated sequence differing from the first time-domain modulated sequence and the second time-domain modulated sequence.

20. The method of claim 19, wherein the second light source is configured to evolve the detectable signal indicative of the presence of the detectable capture moiety but not the second product within the droplet.

21. The method according to claim 1, further comprising capturing a plurality of images of the droplets.

22. The method according to claim 21, further comprising correlating an illumination pattern in the plurality of images to (1) the presence of absence of a detectable capture moiety in a droplet and (2) the presence or absence of the second product in the droplet.

23. The method of claim 22, further comprising correlating the presence or absence of the second product to a condition of a source of the sample.

24. The method of claim 23, wherein the condition is a disease state.

25. A system, the system being configured to perform the method of claim 1.

26. A system, comprising:
a droplet generation section configured to form a plurality of droplets having therein a substrate and a final product comprising an EV and reactive with the substrate to produce a detectable signal indicative of the presence of the final product within the droplet:
an incubation section in fluid communication with the droplet generation section and configured to communicate therein the droplets,
the incubation section operable to provide a residence time for the droplets sufficient to give rise to the detectable signal indicative of the presence of the final product within the droplet: and
a detection section, the detection section configured to communicate therein the plurality of droplets through at least one channel of a microfluidic device, illuminate the droplets with a first time-domain modulated sequence of flashes from a first light source, and capture a plurality of images of the droplets.

27. The system of claim 26, wherein the detection section is configured to illuminate the droplets with a second time-domain modulated sequence of flashes from a second light source

28. The system of claim 26, the system further configured to correlate an illumination pattern in the plurality of images with an expected pattern based at least on the first time domain modulated sequence to determine a number and/or position of droplets containing the final product.

29. The system of claim 26, further comprising a treatment section, the treatment section configured to contact (i) a sample comprising a plurality of extracellular vesicles (EVs) and (ii) a detectable capture modality complementary to a target EV so as to form a first product;

30. The system of claim 29, wherein the treatment section is further configured to contact the pre-product with a detection modality that associates with the target EV so as to form the final product.
